(19) United States
(12) Patent Application Publication
(10) Pub. No.: US 2026/0115230 A1
(43) Pub. Date: Apr. 30, 2026

(54) NEURONAL REGENERATION PROMOTING CELLS (NRPCs) AND TREATING DAMAGED NERVE CELLS

(71) Applicant: CELLATOZ THERAPEUTICS, INC., Seongnam-si (KR)

(72) Inventors: Jaeseung LIM, Hwaseong-si (KR); Ho Jin KIM, Yongin-si (KR); Sung-Chul JUNG, Seoul (KR); Saeyoung PARK, Seoul (KR)

(21) Appl. No.: 19/402,223

(22) Filed: Nov. 26, 2025

Related U.S. Application Data
(63) Continuation of application No. 18/589,244, filed on Feb. 27, 2024.
(60) Provisional application No. 63/462,939, filed on Apr. 28, 2023.

(51) Int. Cl. A61K 35/30; C12N 5/0793

(52) U.S. Cl. CPC A61K 35/30; C12N 5/0619; C12N 2501/115; C12N 2501/13; C12N 2501/135; C12N 2501/195; C12N 2501/41; C12N 2506/1384

(57) ABSTRACT

The present disclosure provides new and innovative compositions of Neuronal regeneration-promoting cells (NRPCs), methods for generating NRPCs, and methods of treating subjects having damaged nerve cells using NRPCs. In an embodiment, the NRPCs are induced from tonsil-derived mesenchymal stem cells and express CD26, CD106, CD112, CD121a, and CD141, wherein CD121a has an expression level of about 30% or higher, the expression level measured immediately after thawing the NRPCs from a frozen state. Furthermore, the NRPCs are configured to promote formation of axons on neuronal cells.

### TECHNICAL FIELD

The present disclosure relates to stem cell-derived, neuronal regeneration-promoting cells (NRPCs), and more specifically to NRPCs having the ability to treat damaged nerve cells and methods thereof.

### BACKGROUND

Neuronal cells, or neurons, form the building block of the nervous system, and transform and relay electrical signals. A neuron may comprise a cell body, dendrites extending from the cell body, and an axon. The axon is a long output structure of the neuron allowing the neuron to propagate the electrical signal as an action potential. Some axons are encased in a fatty substance called myelin, which allows electrical signals to propagate through neurons more effectively. Myelin acts as a form of insulation for axons, helping to send their signals over long distances. The development of myelin around axons, or myelination can be facilitated by neuronal regeneration promoting cells (NRPCs).

Various neurological disorders may result from the improper or insufficient myelination of neuronal cells. For example, Charcot-Marie-Tooth (CMT) disease is a hereditary disease that occurs in 1 in 2,500 people, and the phenotype and genetic causes are heterogeneous. CMT type 1A (CMT1A) is a type of inherited neurological disorder that affects the peripheral nerves and is caused by a duplication of the gene for peripheral myelin protein 22 (PMP22). There is thus a desire and need for an effective treatment of CMT, including CMT1A, where the treatment can facilitate myelination while regulating an overexpression of PMP22.

Mesenchymal stem cells (MSCs) are prevalently used for development of cell therapy agents because they can differentiate into a variety of cell types, in response to specific stimulation. However, the ability for MSCs to effectively develop precursor cells for myelinating neurons is difficult for a variety of factors including, low yield or insufficient neurite growth. There is thus a desire and need for more effective NRPCs and methods of generating the same.

Various embodiments are presented herein that address one or more of these shortcomings.

### SUMMARY

The present inventors discovered that conventional methods of preparing neuronal regeneration promoting cells (NRPCs) from stem cells is very challenging and the NRPCs thus formed may not be as effective in the treatment of damaged nerve cells. The present disclosure provides new and innovative compositions of NRPCs, methods for generating NRPCs, and methods of treating subjects having damaged nerve cells using NRPCs.

Accordingly, in a general embodiment, the present disclosure provides a composition of neuronal regeneration promoting cells (NRPCs). The NRPCs are induced from tonsil-derived mesenchymal stem cells. The NRPCs express CD26, CD106, CD112, CD121a, and CD141. The CD121a has an expression level of 30% or higher.

In an aspect of the present disclosure, which may be combined with any other aspect, the expression level of CD121a in the NRPCs is between about 30% and about 50%, and the expression level of CD121a is measured immediately after thawing the NRPCs from a frozen state.

In an aspect of the present disclosure, which may be combined with any other aspect, the expression level of any given protein (e.g., CD121a) is said to be measured immediately after thawing the NRPCs from the frozen state if the expression level is measured before any subsequent one or more passages after thawing the NRPCs from the frozen state.

In an aspect of the present disclosure, which may be combined with any other aspect, the expression level of CD121a in the NRPCs is about 50% or higher, and the expression level of CD121a is measured one or more passages after thawing the NRPCs from a frozen state.

In an aspect of the present disclosure, which may be combined with any other aspect, the expression level of CD121a in the NRPCs is about 60% or higher, and the expression level of CD121a is measured one or more passages after thawing the NRPCs from a frozen state.

In an aspect of the present disclosure, which may be combined with any other aspect, the expression level of CD121a in the NRPCs is about 70% or higher, and the expression level of CD121a is measured one or more passages after thawing the NRPCs from a frozen state.

In an aspect of the present disclosure, which may be combined with any other aspect, the expression level of CD121a in the NRPCs is about 80% or higher, and the expression level of CD121a is measured one or more passages after thawing the NRPCs from a frozen state.

In an aspect of the present disclosure, which may be combined with any other aspect, the expression level of CD121a in the NRPCs is about 90% or higher, and the expression level of CD121a is measured one or more passages after thawing the NRPCs from a frozen state.

In an aspect of the present disclosure, which may be combined with any other aspect, the expression level of CD26 in the NRPC is 5% or lower, the expression level of CD106 in the NRPC is 15% or higher, the expression level of CD112 in the NRPC is 50% or higher, and the expression level of CD141 in the NRPC is 30% or lower. the expression levels of CD26, CD106, CD112, and CD141 are measured immediately after thawing the NRPCs from a frozen state.

In an aspect of the present disclosure, which may be combined with any other aspect, the expression level of CD26 in the NRPC is 10% or higher, the expression level of CD106 in the NRPC is 10% or higher, wherein the expression level of CD112 in the NRPC is 25% or higher, and the expression level of CD141 in the NRPC is 10% or more. The expression levels of CD26, CD106, CD112, and CD141 are measured one or more passages after thawing the NRPCs from a frozen state.

In an aspect of the present disclosure, which may be combined with any other aspect, the expression level of CD26 in the NRPC is between 10% and 35%, the expression level of CD106 in the NRPC is between 10% and 35%, the expression level of CD112 in the NRPC is between 25% and 90%, and/or the expression level of CD141 in the NRPC is between 10% and 45%. The expression levels of CD26, CD106, CD112, and CD141 are measured one or more passages after thawing the NRPCs from a frozen state.

In a further embodiment, the present disclosure provides a method of producing NRPCs. The method comprises: generating a plurality of cultures of tonsil-derived mesenchymal stem cells (tonsil-derived MSCs) to form neurospheres; generating a plurality of cultures of cells from the neurospheres for inducing into NRPC candidates; and selecting, among the plurality of NRPC candidates, NRPCs that express CD26, CD106, CD112, CD121a, and CD141 and has a first expression level of about 30% or higher for CD121a, the first expression level measured immediately after thawing the NRPC candidates from a frozen state.

In an aspect of the present disclosure, which may be combined with any other aspect, each of the plurality of cultures of tonsil-derived MSCs is generated in a separate container such that each of the containers contains a separate culture comprising tonsil-derived MSCs and a culture medium to form the neurospheres.

In an aspect of the present disclosure, which may be combined with any other aspect, the method further comprises: collecting the neurospheres from each of at least part of the containers containing the neurospheres; and processing the collected neurospheres to further collect cells from the neurospheres.

In an aspect of the present disclosure, which may be combined with any other aspect, each of the plurality of cultures of cells from the neurospheres is generated in a separate container such that each of the containers contains a separate culture comprising the collected cells from the neurospheres and a culture medium for inducing the cells into NRPC candidates.

In an aspect of the present disclosure, which may be combined with any other aspect, a left tonsil tissue and a right tonsil tissue of a single person provide two separate cultures of tonsil-derived MSC.

In an aspect of the present disclosure, which may be combined with any other aspect, the method further comprises: providing the plurality of cultures of tonsil-derived MSCs, which comprises: providing a left tonsil tissue and a right tonsil tissue of a single person; isolating first tonsil-derived MSCs from the left tonsil; and isolating second tonsil-derived MSCs from the right tonsil.

In an aspect of the present disclosure, which may be combined with any other aspect, the method further comprises: the selecting comprises analyzing expression CD markers.

In an aspect of the present disclosure, which may be combined with any other aspect, the selecting comprises performing flow cytometry with regard to one or more of CD26, CD106, CD112, CD121a, or CD141.

In an aspect of the present disclosure, which may be combined with any other aspect, the method further comprises: for each of the plurality of NRPC candidates or a subset thereof, assessing whether the NRPC candidate induces myelination on dorsal root ganglia, wherein selecting selects NRPCs that induce myelination on the Dorsal root ganglia, express the CD26, the CD106, the CD112, the CD121a, and the CD141, and have the first expression level of about 30% or higher for the CD121a, the first expression level measured immediately after thawing the NRPC candidates from the frozen state.

In an aspect of the present disclosure, which may be combined with any other aspect, the assessing comprises: co-culturing dorsal root ganglia and the NRPC candidate subject to assessment; and subsequently examining the dorsal root ganglia and confirming myelination thereon.

In an aspect of the present disclosure, which may be combined with any other aspect, the method further comprises: for each of the plurality of NRPC candidates or a subset thereof, assessing whether the NRPC candidate induces neurite outgrowth on a respective sample of neuroblastoma cells, wherein a given NRPC candidate induces neurite outgrowth if an average number of neurites formed per neuroblastoma cell in the respective sample of neuroblastoma cells is at least 15, and a length of a longest neurite formed in the respective sample of neuroblastoma cells is at least 150 μm; and wherein the selecting further comprises: selecting NRPCs, among the NRPC candidates, that induce the neurite outgrowth, express the CD26, the CD106, the CD112, the CD121a, and the CD141, and has the first expression level of about 30% or higher for the CD121a, the first expression level measured immediately after thawing the NRPC candidates from the frozen state.

In an aspect of the present disclosure, which may be combined with any other aspect, the method further comprises: expanding the selected NRPCs over a plurality of passages; and harvesting NRPCs from at least part of the plurality of passages.

In an aspect of the present disclosure, which may be combined with any other aspect, the method further comprises: discarding at least one NRPC candidate that has a first expression level for CD121a less than 30%.

In an aspect of the present disclosure, which may be combined with any other aspect, NRPCs having a second expression level of about 75% or higher for CD121a are selected, the second expression level measured one or more passages after thawing the NRPC candidates from a frozen state.

In an aspect of the present disclosure, which may be combined with any other aspect, NRPCs having a second expression level of about 80% or higher for CD121a are selected, the second expression level measured one or more passages after thawing the NRPC candidates from a frozen state.

In an aspect of the present disclosure, which may be combined with any other aspect, NRPCs having a second expression level of about 85% or higher for CD121a are selected, the second expression level measured one or more passages after thawing the NRPC candidates from a frozen state.

In an aspect of the present disclosure, which may be combined with any other aspect, NRPCs having a second expression level of about 90% or higher for CD121a are selected, the second expression level measured one or more passages after thawing the NRPC candidates from a frozen state.

In an aspect of the present disclosure, which may be combined with any other aspect, NRPCs having a third expression level of about 5% or lower for CD26 are selected, NRPCs having a fourth expression level of about 15% or higher for CD106 are selected, NRPCs having a fifth expression level of about 50% or higher for CD112 are selected, and NRPCs having a sixth expression level of about 30% or lower for CD141 are selected. The third, fourth, fifth, and sixth expression levels are measured immediately after thawing the NRPC candidates from the frozen state.

In an aspect of the present disclosure, which may be combined with any other aspect, NRPCs having a seventh expression level of about 10% or higher for CD26 are selected, NRPCs having a eighth expression level of about 10% or higher for CD106 are selected, NRPCs having a ninth expression level of about 25% or higher for CD112 are selected, and NRPCs having a tenth expression level of about 10% or higher for CD141 are selected. The seventh, eighth, ninth, and tenth expression levels are measured one or more passages after thawing the NRPC candidates from the frozen state.

In an aspect of the present disclosure, which may be combined with any other aspect, NRPCs having the seventh expression level between about 10% and about 35% for CD26 are selected, NRPCs having the eighth expression level between about 10% and about 35% for CD106 are selected, NRPCs having the ninth expression level between about 25% and about 90% for CD112 are selected, and NRPCs having the tenth expression level between about 10% and about 45% for CD141 are selected. The seventh, eighth, ninth, and tenth expression levels are measured one or more passages after thawing the NRPC candidates from the frozen state.

In a further embodiment, the present disclosure provides a method of treating damaged nerve cells, the method comprising: administration, into a subject's body having damaged nerve cells, any of the compositions comprising NRPCs described herein in an effective amount for causing myelination of the damaged nerve cells or remyelination of Schwann cells.

In a further embodiment, the present disclosure provides a method of treating muscle fibrosis, the method comprising: administration, into a subject's body having muscle fibrosis, any of the compositions comprising NRPCs described herein in an effective amount for treating the muscle fibrosis.

In a further embodiment, the present disclosure provides a method of treating muscle inflammation, the method comprising: administration, into a subject's body having muscle inflammation, any of the compositions comprising NRPCs described herein in an effective amount for treating the muscle inflammation.

In a further embodiment, the present disclosure provides a method of causing vascularization in an ischemic tissue, the method comprising: administration, into a subject's body having an ischemic tissue, any of the compositions comprising NRPCs described herein in an effective amount for causing vascularization.

In a further embodiment, the present disclosure provides a method of treating critical limb ischemia (CLI), the method comprising: injecting, into a subject's body having the CLI, any of the compositions comprising NRPC described herein in an effective amount for treating the CLI.

In a further embodiment, the present disclosure provides a method of treating peripheral nerve damage, the method comprising: administrating, into a subject's body having the peripheral nerve damage, any of the compositions comprising NRPCs described herein in an effective amount for treating the peripheral nerve damage.

In a further embodiment, the present disclosure provides a method of suppressing overexpression of peripheral myelin protein 22 (PMP22) in a subject. The method comprises: administering, into a localized area of a subject's body in which overexpression of PMP22 is confirmed or assessed, a composition comprising NRPCs described herein in an effective amount for suppressing the overexpression of PMP22 at least in the localized area.

In an aspect of the present disclosure, which may be combined with any other aspect, the method treats Charcot-Marie-Tooth (CMT) in the subject.

In an aspect of the disclosure, which may be combined with any other aspects, the composition comprising NRPCs described herein is administered into a subject's body in an effective amount. In an embodiment the composition being administered is in a frozen state. In another embodiment, the composition is administered one or more passages after thawing the NRPCs from a frozen state.

In a further embodiment, the present disclosure provides a method of increasing expression of miR-29a. The method comprises: administering, into a localized area of a subject's body in which a need for increasing expression of miR-29a is confirmed or assessed, a composition comprising NRPCs described herein in an effective amount for increasing the expression of miR-29a at least in the localized area.

In an aspect of the disclosure, which may be combined with any other aspects, increasing the expression of miR-29a causes suppressing overexpression of peripheral myelin protein 22 (PMP22) at least in the localized area.

In an aspect of the disclosure, which may be combined with any other aspects, the method treats Charcot-Marie-Tooth (CMT) in the subject.

In the present disclosure, the term “subject” refers to an individual requiring the administration of the composition or the neuronal regeneration-promoting cells of the present disclosure, and includes mammals (e.g., humans), birds, reptiles, amphibians, fish, etc. without limitation.

In the present disclosure, “treatment” refers to any action of improving or favorably changing the symptoms of a disease by administering the composition according to the present disclosure.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE FIGURES

FIG. 1 illustrates population doubling times and cell sizes of different mesenchymal stem cells (MSCs) derived from various regions, according to an example embodiment of the present disclosure.

FIG. 2 illustrates cell size, viability, and doubling time for tonsil-derived mesenchymal stem cells (TMSCs) obtained from different humans, according to an example embodiment of the present disclosure.

FIG. 3 illustrates marker expression rate for MSCs derived from different regions of a human, and neuronal regeneration promoting cells (NRPCs) differentiated from the MSCs, according to an example embodiment of the present disclosure.

FIG. 4A and FIG. 4B show the cytokine expression rate among MSCs and NRPCs derived from different regions of a human, according to an example embodiment of the present disclosure.

FIG. 5 is a set of immunofluorescence images showing the increased expression of proteins relevant for nerve health for the NRPCs obtained from different stages of the NRPC production process, according to an example embodiment of the present disclosure.

FIGS. 6A and 6B show different levels of neurite outgrowth among MSCs and NRPCs derived from different regions of a human, according to an example embodiment of the present disclosure.

FIG. 7 is a set of images showing different neurite outgrowth among samples of tonsil-derived MSCs (T-MSCs) and tonsil-derived NRPCs (T-NRPCs), according to an example embodiment of the present disclosure.

FIGS. 8A and 8B show a set of graphs showing the average number of neurites and the average length of neurites for neurite growth assays performed among samples of tonsil-derived MSCs (T-MSCs) and tonsil-derived NRPCs (T-NRPCs), according to an example embodiment of the present disclosure.

FIGS. 9A and 9B show a set of graphs and images showing decreasing neurite growth among samples of T-MSCs and T-NRPCs when the expression of the CD121a was reduced using small interfering RNA (siRNA), according to an example embodiment of the present disclosure.

FIGS. 10A and 10B shows expression levels of the marker CD121a among working cell banks of MSCs and NRPCs derived from various regions, and among the NRPCs, according to an example embodiment of the present disclosure.

FIGS. 11A and 11B show the expression levels of the marker CD121a among T-MSCs and NRPCs through various passages, according to an example embodiment of the present disclosure.

FIG. 12 is a set of images and graphs showing a correlation between neurite outgrowth assay and expression levels of the marker CD121a among of T-MSCs and NRPCs through various passages, according to an example embodiment of the present disclosure.

FIGS. 13A and 13B show a set of graphs showing the average number of neurites and the average length of neurites for T-MSCs and NRPCs through various passages, according to an example embodiment of the present disclosure.

FIGS. 14A, 14B and 14C show a set of graphs showing the expression of CD121a in T-MSCs 2009R and NRPCs 2009R corresponding to passages 15 to 19 according to an example embodiment of the present disclosure.

FIGS. 15A and 15B show a set of images showing the differentiation of MSCs, derived from various regions of a human, to NRPCs, and a set of graphs indicating the expression rate of the marker CD121a among NRPCs and their corresponding MSCs derived from various regions of a human, according to an example embodiment of the present disclosure.

FIGS. 16A and 16B show expression levels of the marker CD121a among working cell banks of MSCs and NRPCs derived from various regions, and among the NRPCs, according to an example embodiment of the present disclosure.

FIGS. 17A, 17B and 17C show blood flow analysis over a period of time in animal samples having critical limb ischemia (CLI) and undergoing various forms of treatment based on MSCs and NRPCs, according to an example embodiment of the present disclosure.

FIGS. 18A and 18B show sets of images illustrating muscle fibrosis, muscle inflammation, and capillary formation over a period of time in animal samples receiving various forms of treatment, according to an example embodiment of the present disclosure.

FIGS. 19A, 19B and 19C show the results of a nerve conduction study performed on mice samples using varying levels of T-NRPCs, according to an example embodiment of the present disclosure.

FIGS. 20A, 20B and 20C show three sets of images of immunochemical stained sciatic nerves of mice samples treated using varying levels of T-NRPCs to show expression of various protein markers, according to an example embodiment of the present disclosure.

FIGS. 21A to 21E shows five sets of images indicating the G-ratio and myelination of neurons of mice samples treated using varying levels of T-NRPCs, according to an example embodiment of the present disclosure.

FIGS. 22A to 22D show three sets of images and a graph indicating the expression of markers PMP22 and MPZ among mice samples treated using varying levels of T-NRPCs, according to an example embodiment of the present disclosure.

FIGS. 23A and 23B show a table illustrating the analysis of several miRNAs to determine which appeared to be expressed in TMSC and T-NRPC cultures, according to an example embodiment of the present disclosure.

### DETAILED DESCRIPTION OF EXAMPLE EMBODIMENTS

The presently disclosed subject matter now will be described and discussed in more detail in terms of some specific embodiments and examples with reference to the accompanying drawings, in which some, but not all embodiments of the invention are shown. Like numbers refer to like elements or parts throughout. The presently disclosed subject matter may be embodied in many different forms and should not be construed as limited to the specific embodiments set forth herein. Rather, these embodiments are provided so that this disclosure will satisfy applicable legal requirements. Indeed many modifications and other embodiments of the presently disclosed subject matter will come to the mind of one skilled in the art to which the presently disclosed subject matter pertains. Therefore, it is to be understood that the presently disclosed subject matter is not to be limited to the specific embodiments disclosed and that modifications and other embodiments are intended to be included within the scope of the appended claims.

### Various Neurological Disorders Arise from Improper or Insufficient Myelination

Myelin is a lipid-rich material that surrounds the axons of neuronal cells (e.g., as myelin sheaths) to insulate them and increase the rate at which action potentials are propagated along the axon. Myelin is formed by glial cells such as oligodendrocytes and Schwann cells. Various neurological disorders may result from improper or insufficient myelination of neuronal cells or insufficient growth of axons and other neurites. For example, Charcot-Marie-Tooth (CMT) disease is a hereditary disease that occurs in 1 in 2,500 people, and the phenotype and genetic causes are heterogeneous. CMT type 1A (CMT1A) is a type of inherited neurological disorder that affects the peripheral nerves and is caused by a duplication of the gene for peripheral myelin protein 22 (PMP22).

### Such Neurological Disorders Manifest a Need for More Effective Stem Cell Treatment

Damaged nerves, whether through improper or insufficient myelination, or insufficient neurite growth, may be replaced or regenerated using stem cells. In particular, mesenchymal stem cells (MSCs) are prevalently used for development of cell therapy agents because they can differentiate into a variety of cell types, in response to specific stimulation. The cell types relevant for recovering damaged nerves may require the effective expression of proteins responsible for replacing, regenerating, and/or myelinating nerves. However, the ability for MSCs to effectively differentiate into the appropriate precursor cells (referred to herein as “neuronal regeneration promoting cells (NRPCs)”), which then express the proteins responsible for replacing, regenerating, and/or myelinating nerves, is difficult for a variety of factors. For example, there is often low yield for such proteins expressed by the NRPCs derived from the differentiated MSCs, or the NRPC samples may yield insufficient neurite growth.

### The Present Disclosure Describes a New and Improved NRPCs for Treating Damaged Neurons, and Methods of Generating Thereof

The present disclosure describes more effective NRPCs for treating damaged neurons and methods of generating such NRPCs. The inventors of the present disclosure have discovered that NRPCs differentiated from MSCs obtained from certain regions of a human (e.g., tonsils) appear to have better rates of expression of relevant proteins for treating damaged nerves for the aforementioned neurological disorders. Such MSCs may be referred to herein as tonsil-derived MSCs (T-MSCs). Furthermore, the inventors have discovered that NRPCs derived from T-MSCs (referred to herein as T-NRPCs) that express the protein markers CD26, CD106, CD112, CD121a, and CD141 have a higher success at treating damaged nerves, and a method of selectively expanding such T-NRPCs expressing such proteins is disclosed. In particular, the inventors have discovered that selectively expanding T-NRPCs expressing the protein marker CD121a above threshold levels (e.g., above 30% at the working cell bank (WCB) stage) greatly increase the effectiveness of NRPCs for treating damaged nerves. Furthermore, the inventors have discovered that selectively screening and expanding T-NRPC samples for those that exhibit neurite formation leads to more effective products for the treatment of damaged nerves.

### The Origin of the MSCs is Relevant for Generating Effective NRPCs

As will be discussed herein in conjunction with experimental data described herein, the inventors have discovered that the origin of the MSCs is relevant for generating effective NRPCs. For example, the region of the human body from which MSCs are harvested have an effect on the ability of the MSCs to differentiate to NRPCs effectively and/or express relevant protein markers effectively.

### Although MSCs from Various Regions of a Human can Express Relevant Protein Markers Identically, the Population Doubling Time Tends to Vary Among MSCs

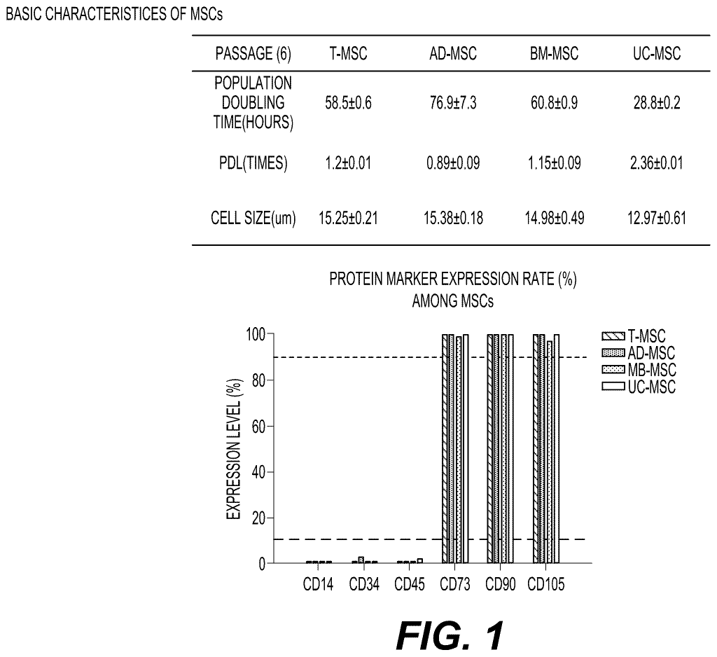

For example, FIG. 1 illustrates population doubling times and cell sizes of different samples of mesenchymal stem cells (MSCs) derived from various regions of a human body, according to an example embodiment of the present disclosure. The various regions include the tonsils, adipose tissue, bone marrow, and umbilical cord, from which tonsil derived MSCs (T-MSCs), adipose-derived MSCs (AD-MSCs), bone marrow-derived MSCs (BM-MSCs), and umbilical cord derived MSCs (UC-MSCs) are obtained, respectively. Generating NRPCs effectively from MSCs depends on the ability of the MSCs to grow in population size in order to allow sufficient amount of MSCs to differentiate into a desired NRPC candidate cells. The growth in population size of any cell sample can be measured by determining the cell sample's population doubling time, which is the time (e.g., hours) it takes for a cell sample's cell count to double. Also or alternatively, the growth can be measured by the determining the cell sample's population doubling level (PDL), which is the total number of times the cells in a given population have doubled during in vitro culture. As shown in FIG. 1, the growth appears to vary among the different MSC samples. For example, based on population doubling time and population doubling levels, UC-MSCs and T-MSCs were found to have the highest growth in population size. However, irrespective of growth rate, it was found that the expression levels for characteristic markers indicating that the cells were MSCs—the markers being CD73, CD90, and CD105—were roughly the same across each of the four MSC samples. The expression levels confirmed the presence of MSCs in the harvested samples from the four regions (tonsils, adipose tissue, bone marrow, and umbilical cord) of the human. Although the expression levels of such proteins confirmed that the obtained cell samples indeed contained MSCs, expression levels of other proteins indicate that T-MSCs compared to other MSCs generate more effective NRPCs, as will be discussed herein.

### The Cell Size, Cell Viability, and Population Doubling Time was Measured for the MSCs Obtained from the Tonsil Regions of Different Humans

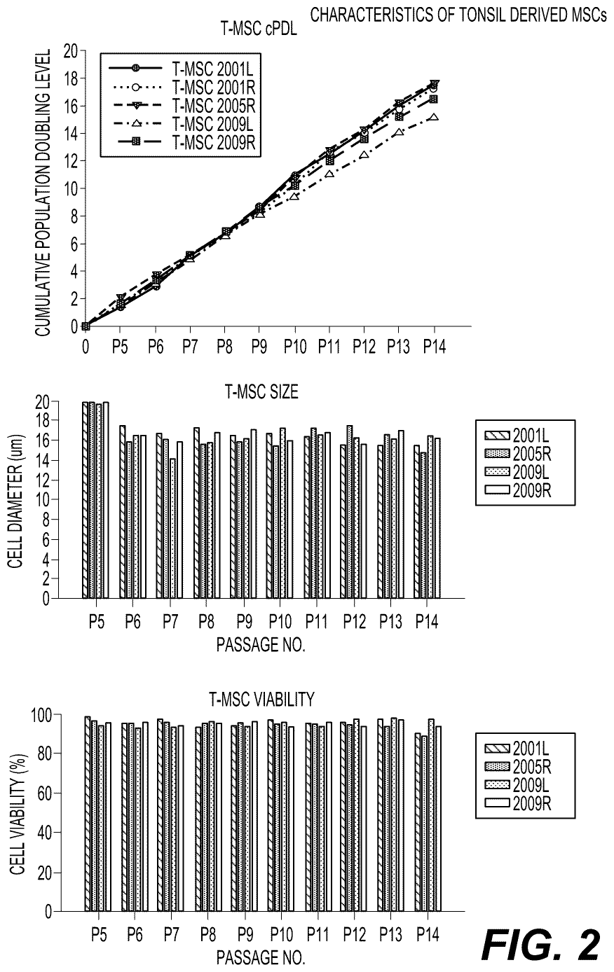

As previously discussed, the origin of the MSCs is relevant for generating effective NRPCs, since the particular region of the human from which MSCs are obtained (e.g., tonsil, adipose tissue, bone marrow, umbilical cord, etc.) can be dispositive for the effectiveness of NRPCs and/or MSCs used in producing the NRPCs. Specifically, as will be described in the studies below, the inventors discovered that the tonsil-derived MSCs were the most effective at generating NRPCs (T-NRPCs) with the best results for nerve regeneration (e.g., as measured by neurite outgrowth). In order to begin the studies, the inventors obtained MSCs from the same region (e.g., tonsils) of different humans. However, the inventors tested the obtained MSCs to make sure they were consistent with respect to cell size, cell viability, and population doubling time. The inventors contemplated that differences in cell size, cell viability, and population doubling time could impact the effectiveness of the study (e.g., by introducing unintended variables), so an assessment for consistency within the obtained samples of T-MSCs was performed. FIG. 2 illustrates cell size, viability, and doubling time for tonsil-derived mesenchymal stem cells (T-MSCs) obtained from different humans. The T-MSCs are identified as 2001L, 2001R, 2005R, 2009L, and 2009R, each identification indicating the human (e.g., by numbers 2001, 2005, 2009, etc.) and the left or right tonsil from which the T-MSCs are obtained (e.g., L or R). Specifically, the population doubling level, cell size, and cell viability were measured for T-MSC samples obtained from at least the humans identified as 2001L, 2005R, 2009L, and 2009R across passages. The population doubling level appeared to increase for all T-MSC samples as the number of passages increased, but T-MSC 2009L samples from human 2009L increased at a lower rate. The cell size, measured by cell diameter (in micrometers), appeared to remain the same for the T-MSC samples. The cell viability, which was measured by the trypan blue staining method, appeared to remain the same for all of the T-MSC samples. Nevertheless, there were no significant differences in cell size and viability, and population doubling time at least among the samples for any given passage. Therefore, the inventors were able to ensure the consistency of the samples of T-MSCs obtained from the humans.

### The Use of Passages in the Processes Described Herein

As used herein, a passage may indicate the process where cell culture from a sample is subcultured, i.e. harvested and reseeded into one or more ‘daughter’ cell culture flasks, in order for the reseeded cells to develop into cell cultures again in their respective daughter cell culture flasks. The process may be iterated, such that cell culture from the ‘daughter’ cell culture flasks can be subcultured again in subsequent ‘daughter’ cell culture flasks (or other suitable container). A passage number may indicate the number of these iterations. For example, passage 5 (P5) indicates that cells from an existing cell culture, has been harvested and reseeded into a new cell culture flask (to develop into a new cell culture again) five times. As described herein, the methods of producing the disclosed NRPCs for the treatment of damaged nerves may involve culturing and subculturing cell samples (e.g., of MSCs, candidate NRPCs, and NRPCs) over many passages. By doing so, each subsequent cell culture is replenished with fresh growth medium, thereby optimizing the expansion, health, and stability of cell cultures.

### The Expression Rate for CD121a Varies Based on the Origin of the MSC

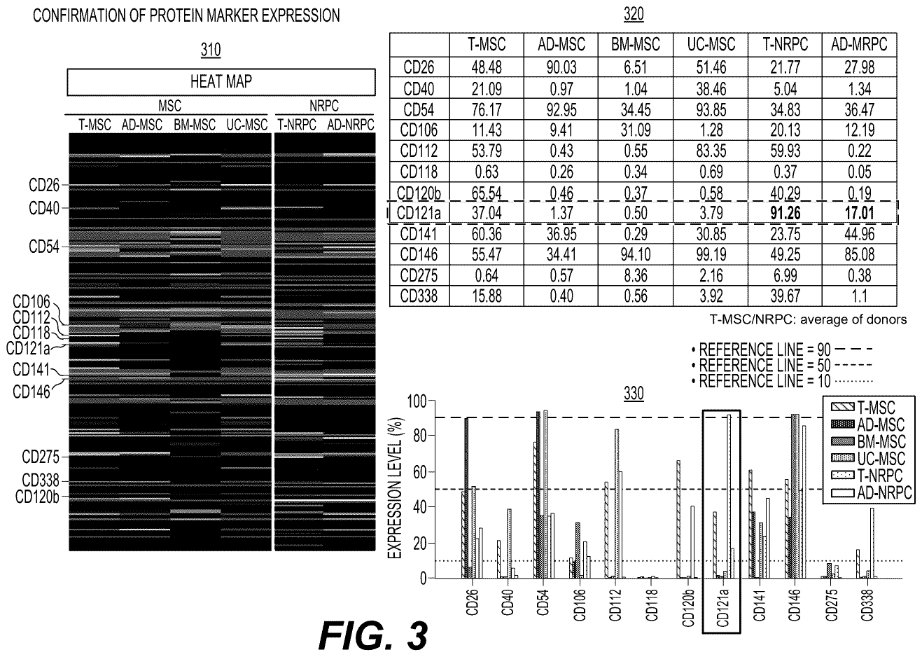

As previously discussed, the inventors discovered that new and improved NRPCs for treating damaged cells can be generated by selectively screening for NRPCs expressing certain marker proteins, particularly CD121a. The inventors have also found that the expression rate for CD121a varies based on the origin of the MSCs that differentiate to the NRPCs. FIG. 3 illustrates marker expression rate for MSCs derived from various regions of a human, and NRPCs differentiated from the MSCs, according to an example embodiment of the present disclosure. The expression rates are illustrated through a heat map 310, a table 320, and a histogram chart 330. The heat map 310 indicates, in the intensity of a red color, the expression rate for the various marker proteins relevant for the treatment of damaged nerves, including CD121a. The heat map 310 confirms that expression rates for the marker proteins differ depending on the origin of the MSC samples, and that the marker expression rates also differ among the differentiated NRPC samples. The table 320 lists, and the histogram 330 illustrates (using the height of the bars), the actual expression rates for the various marker proteins relevant for the treatment of damaged nerve cells among the MSC and NRPC samples, which vary based on the origin of the sample. In particular, the table 320 and histogram 330 show that the expression rate for CD121a is higher in T-MSCs than in MSCs originating from other tissues (AD-MSCs, BM-MSCs, and UC-MSCs). Furthermore, the table 320 and histogram 330 show that the expression rate for CD121a is much higher in T-NRPCs than in AD-NRPCs. The inventors have discovered that by selectively screening, culturing, and expanding MSCs and/or T-NRPCs based on a selection criteria defined by the expression rate for relevant marker proteins, particularly CD121a, the resulting T-NRPC products are significantly more effective at treating damaged nerve cells. Based on the experimental results shown in FIG. 3, CD121a is expressed at 91% among the T-NRPCs but only expressed at 17% among the AD-NRPCs (an expression level of about 17% may be below a threshold for selection, and therefore such samples may be considered as “non-selectable” as will be described below). The expression rate for CD121a is also different among each MSC sample. The expression rate of CD121a among the T-MSCs is higher than that of the other MSCs. When MSCs of each region differentiated into their respective NRPCs, the expression rates similarly increased, but the rate of increase varied based on the origin of the MSCs. In the case of AD-MSCs originating from adipose tissue, the increase in expression rate was seen from 1% (among AD-MSCs) to 17% (among AD-NRPCs), but the increase in expression rate was significantly lower than that of T-MSCs originating from tonsils, where the increase in expression rate was from 37% (among T-MSCs) to 91% (among T-NRPCs). The experimental results show that T-NRPCs can be identified and selected based on high expression rates in expressing CD121a.

### Selectable Versus Non-Selectable Samples Based on Expression Rates for Protein Markers Meeting or not Meeting Expression Rate Thresholds, Respectively

As discussed herein, cell samples (e.g., NPRCs and/or NRPC candidates) may be selected (e.g., deemed as “selectable”) or may not be selected (e.g., deemed as “non-selectable”) based on a cell sample's expression rate for a protein marker of interest. Depending on the protein marker, different thresholds may be established for the expression rate for that protein marker. If the expression rate for a given protein marker by a given cell sample is above a threshold expression rate assigned for that given protein marker, the cell sample is deemed as a selectable cell sample. However, if the expression rate for the given protein marker by the given cell sample is below the threshold expression rate assigned for that given protein marker, the cell sample is deemed as a non-selectable cell sample. Furthermore, a cell's ability to express a protein may depend on the state of the cell at which an expression level of the protein is measured.

For example, the cells may be in a frozen state, for which an expression level for a protein in the cells in the frozen state can be determined and/or deduced by measuring the expression level for the protein in the cells immediately after thawing the cells from the frozen state. By measuring the expression level at a time immediately after thawing, heat-dependent cell activities (e.g., enzymatic activities) that could affect the expression rate can be avoided. The duration of time after thawing that can be considered to be “immediately after thawing” can be within about 0.2, 0.4, 0.6, 0.8, 1, 1.5, 2, 2.5, 3, 3.5, 4, 4.5, 5, 5.5, 6, 6.5, 7, 8, 9, 10, 12, 14, 16, 18, 20, 25, 30, 35, 40, 45, 50, 55, or 60 minute after thawing. In embodiments, the time after thawing that forms the upper limit for what is considered immediately after thawing may be within a range formed by selecting any two numbers (two times) listed in the immediately previous sentence (e.g., the time after thawing that forms the upper limit for what is considered immediately after thawing may be between about 1 minute and about 5 minutes, between about 30 seconds and about 10 minutes, between about 2 minutes and about 4 minutes, etc.). Also or alternatively, a time “immediately after thawing” the cells may be before any passages after thawing the cells. In embodiments, the threshold for a cell sample to be selectable, based on an expression rate or level at which a protein marker is expressed, may be higher for cells that are in a non-frozen or a “live” state (e.g., as a product) than for cell in a frozen state. Cells that are in a non-frozen or “live” state may comprise cells that are one or more passages after being thawed from a frozen state. It is contemplated that other protein markers may have different (e.g., lower or higher) thresholds for expression rates to determine whether cells expressing such protein markers are selectable or non-selectable. In some embodiments, cells that are in a non-frozen or a live state may not have been in a frozen state previously, and thus may not require any thawing process to be performed before any one or more passages prior to measurement of any protein expression rate.

### Selectability May Depend on a First Threshold for CD121a Expression at an Earlier Stage in the NRPC Production Process

For example, in at least some embodiments, cell samples may be selected (e.g., deemed “selectable”) if they express CD121a at an expression rate above a first threshold (e.g., 30%) when the cells are in a frozen state (e.g., during the working cell bank stage) but above a second threshold (e.g., 80%) when the cells are in a non-frozen or live state (e.g., during the product stage). The different thresholds for protein expression required for the selection of a cell sample may depend on the stage that the cell sample is in the NRPC production process, such that a lower threshold may be required for protein expression if the cell sample is at an earlier stage, but a higher threshold may be required for protein expression if the cell sample is at a later stage. In particular, a lower threshold for CD121a expression may be set to select and expand cell samples at the working cell bank stage. The cell samples being selected at this earlier stage (referred to herein as “NRPC-candidates”) may be in a frozen state and may comprise MSCs in the process of differentiating into respective NRPCs. Also or alternatively, the NRPC-candidates may have already differentiated into NRPCs but may require further development and/or differentiation. For example, the inventors found that NRPC candidates in the working cell bank stage having a CD121a expression level higher than a first threshold rate of about 30% were able to ultimately differentiate into the desired NRPC samples able to express CD121a above a second threshold at a later stage (e.g. product stage). However, in some embodiments, the first threshold for CD121a expression level at this earlier stage may be higher or lower. For example, NRPC-candidates may be selected if their CD121a expression levels are at least above a threshold, which is about 25%, 27.5%, 30%, 32.5, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, or 80%. In embodiments, the threshold may be within a range formed by selecting any two numbers (two percentages) listed in the immediately previous sentence (e.g., an NRPC candidate sample may be selected if the CD121a expression rate that is at least above a threshold, where the threshold is about 30%, between about 30% and about 40%, between about 35% and about 50%, etc.).

### Selectability May Also Depend on a Second Threshold for CD121a Expression at a Later Stage in the NRPC Production Process

A second threshold for CD121a expression may be used to select, from the products of NRPCs, which may be in a live or non-frozen state, as previously discussed. For example, the inventors found that NRPC candidates in the product stage, one or more passages after thawing, which had a CD121a expression level higher than a second threshold rate of about 80% were able to ultimately form the improved NRPCs that provided the advantages described herein. However, in some embodiments, the second threshold for CD121a expression level at this later stage may be higher or lower. For example, for NRPCs in the later stage (e.g., a product stage and/or a “live” state), NRPCs may be selectable if they express CD121a at an expression rate that is at least above a second threshold, which is about 70%, 72.5%, 75%, 77.5%, 80%, 82.5%, 85%, 87.5%, 90%, 92.5%, 95%, 97.5%, or 100%. In embodiments, the threshold may be within a range formed by selecting any two numbers (two percentages) listed in the immediately previous sentence (e.g., a cell is selectable if it expresses CD121a at an expression rate that is at least above a threshold, where the threshold is about 70%, between about 72.5% and about 77.5%, between about 85% and about 95%, etc.).

### Some MSCs and NRPCs were Non-Selectable Because they Did not Satisfy Expression Rate Threshold for CD121a

FIG. 3 shows the expression rates of various protein markers, including CD121a, as MSCs and NRPCs. As shown in FIG. 3, expression rates for CD121a increased among some cell sample as they differentiated from MSCs to NRPCs. For example, the expression rate for CD121a by adipose tissue derived NRPCs (AD-NRPCs) were higher than adipose derived MSCs (AD-MSCs), from which the AD-NRPCs differentiated. However, despite the increase, the CD121a expression rate were not sufficiently substantial enough for the AD-MSCs or the AD-NRPCs to be considered selectable. As shown in the table 320 and histogram 330 of FIG. 3, the AD-MSC, bone marrow derived MSC (BM-MSC), and umbilical cord derived MSC (UC-MSC) samples also failed to express the CD121a protein marker at sufficiently high expression rates (e.g., above about 30%) in the earlier stages, and were therefore non-selectable. In contrast, the expression of CD121a was higher in tonsil-derived MSCs (T-MSCs) than in MSCs derived from other tissues. The inventors found that T-MSC samples were able to satisfy a first threshold (e.g., about 30%) by expressing CD121a at 37.04%, and were thus considered selectable. The inventors also found that the CD121a expression rate increased as T-MSCs were differentiated into their respective NRPCs (T-NRPCs). After selectively differentiating the T-MSC samples to their respective T-NRPCs, samples of live T-NRPCs obtained at the product stage (e.g., after a first subculture (e.g., passage) after thawing) were also able to successfully express the CD121a protein marker above a second threshold (e.g., of about 80%) by expressing CD121a at 91.26%, and were therefore also deemed selectable.

### The Expression Rates of Other Proteins—CD26, CD106, CD112, and CD141—were Also Relevant

Although it was noted that CD121a was expressed by T-NRPC at a significantly high level, FIG. 3 also shows other relevant trends in the expression rates of proteins as different MSCs differentiated into their respective NRPCs. For example, when protein marker expression patterns of the MSCs are compared with the protein marker expression patterns of the respective NRPCs to which the MSCs differentiated into, certain CD markers are relevant for exhibiting a significant increase or decrease in expression rates as MSCs differentiated into their respective NRPCs. As shown in FIG. 3, the CD markers for which the expression in the NRPCs increased (when compared to the respective MSCs from which the NRPCs were derived from), included CD106 and CD112 (in addition to CD121a described above). The increased expression was most prominent for the tonsil derived NRPCs (T-NRPCs) differentiated from the tonsil-derived MSCs (T-MSCs). Furthermore, as shown in FIG. 3, the CD markers for which the expression in the NRPCs decreased (when compared to the respective MSCs from which the NRPCs were derived from) included CD26 and CD141. The pattern of these CD markers, the expression of which has increased (e.g., CD106, CD112, and CD121a) or decreased (CD26 and CD141), are thus useful for identifying NRPCs differentiated from MSCs. While the markers CD121a, CD106, CD112, CD26 and CD141, the expression of which has changed commonly, can be used as differentiation markers of neuronal regeneration-promoting cells, the significantly high expression rates for CD121a is particularly useful for identifying effective NRPCs, as discussed herein.

### NRPCs can be Selected Based on Expression Levels of CD106, CD112, and CD121a when the NRPCs or NRPC-Candidates are in the Product Stage

The increased expression rates of CD106, CD112 and CD121a and the decreased expression rates of CD26 and CD141 can be identified to select NRPCs differentiated from MSCs. As shown in the specific example of FIG. 3, the T-NRPCs expressed CD106 at 20.13%, CD112 at 59.93%, and CD121a at 91.26% based on a measurement of the expression levels of the aforementioned proteins when the T-NRPCs were in a live unfrozen state in the product stage (e.g., after one passage after thawing). In some embodiments, NRPCs may be selected based on the expression rate for CD106 being at least above a threshold, which is about 10%, 12.5%, 15%, 17.5%, 20%, 22.5%, 25%, 27.5%, 30%, 32.5%, 35%, 37.5%, or 40%. In embodiments, the threshold may be within a range formed by selecting any two numbers (two percentages) listed in the immediately previous sentence (e.g., the NRPC may be selected if the expression rate for CD106 is at least above about 10%, between about 10% and about 30%, between about 15% and about 25%, etc.) In some embodiments, NRPCs may be selected based on the expression rate for CD112 being at least above a threshold, which is about 25%, 30%, 35%, 40%, 50%, 60%, 70%, 80%, or 90%. In embodiments, the threshold may be within a range formed by selecting any two numbers (two percentages) listed in the immediately previous sentence (e.g., the NRPC may be selected if the expression rate for CD112 is at least above about 25%, between about 25% and about 40%, between about 30% and about 50%, etc.). As previously discussed, effective T-NRPCs can be identified and selected based on high expression rates in expressing CD121a. For example, T-NRPCs may be identified and selected based on the expression rate for CD121a, while the T-NRPCs are in an nonfrozen state (e.g., one or more passages after thawing), being at least above a threshold. The threshold may be about 80%, 82.5%, 85%, 87.5%, 90%, 92.5%, 95%, 97.5%, or 100%. In embodiments, the threshold may be within a range formed by selecting any two numbers (two percentages) listed in the immediately previous sentence (e.g., T-NRPCs may be identified and selected when CD121a is expressed at least above a threshold, where the threshold is about 80%, between about 82.5% and about 87.5%, between about 85% and about 95%, etc.).

### NRPCs can be Selected Based on Expression Levels of CD106, CD112, and CD121a when the NRPC or NRPC-Candidates are in their Working Cell Bank Stage

In some embodiments, T-NRPCs can be selected based on expressions rates when the T-NRPCs or T-NRPC candidates are in a working cell bank stage (e.g., immediately after thawing from a frozen state (e.g., before any subsequent passages after thawing)). As previously discussed, expression rates for a protein in cells that are in a frozen state can be determined and/or deduced by measuring the expression rates for the protein in the cells immediately after thawing the cells from the frozen state. By measuring the expression rates at a time immediately after thawing, heat-dependent cell activities (e.g., enzymatic activities) that could affect the expression rates can be avoided. The duration of time after thawing that can be considered to be “immediately after thawing” can be within about 0.2, 0.4, 0.6, 0.8, 1, 1.5, 2, 2.5, 3, 3.5, 4, 4.5, 5, 5.5, 6, 6.5, 7, 8, 9, 10, 12, 14, 16, 18, 20, 25, 30, 35, 40, 45, 50, 55, or 60 minute after thawing. In embodiments, the time after thawing that forms the upper limit for what is considered immediately after thawing may be within a range formed by selecting any two numbers (two times) listed in the immediately previous sentence (e.g., the time after thawing that forms the upper limit for what is considered immediately after thawing may be between about 1 minute and about 5 minutes, between about 30 seconds and about 10 minutes, between about 2 minutes and about 4 minutes, etc.). For example, T-NRPCs may be identified and selected based on the expression rate for CD106 being at least above a threshold, which may be about 15%, 17.5%, 20%, 22.5%, 25%, 27.5%, 30%, 32.5%, or 35%, the expression rate for CD106 being measured when the T-NRPC or T-NRPC candidate is at the working cell bank stage. T-NRPCs may be identified and selected based on the expression rate for CD112 being at least below a threshold, which may be about 50%, 52.5%, 55%, 57.5%, 60%, 62.5%, 65%, 67.5%, or 70%, the expression rate for CD112 being measured when the T-NRPC or T-NRPC candidate is at the working cell bank stage. As previously discussed, T-NRPC or T-NRPC candidates can also be selected based on threshold expression levels of CD121a when the T-NRPC or T-NRPC candidates are in the working cell bank stage. For example, T-NRPCs or T-NRPC-candidates may be selected if their CD121a expression levels are at least above a threshold, which is about 25%, 27.5%, 30%, 32.5, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, or 80%. In embodiments, the threshold may be within a range formed by selecting any two numbers (two percentages) listed in the immediately previous sentence (e.g., an NRPC candidate sample may be selected if the CD121a expression rate that is at least above a threshold, where the threshold is about 30%, between about 30% and about 40%, between about 35% and about 50%, etc.).

### NRPCs can be Selected Based on Expression Levels of CD26 and CD141 when the NRPC or NRPC-Candidates are in their Product Stage

T-NRPCs can be selected based on measuring expression levels for CD26 and CD141, when the T-NRPCs are in their product stage (e.g., in a live, unfrozen state one or more passages after thawing). For example, in the specific experiment shown in FIG. 3, the T-NRPCs are selected based on expression rates for CD26 at 21.77% and CD141 at 23.75%.

In some embodiments, T-NRPCs may be identified and selected based on the expression rate for CD26 being at least above a threshold, which may be about 4, 5, 7, 8, 9, 10, 11, 12, 13, 14, 15, 16, 17, 18, 19, 20, 21, 22, 23, 24, 25, 26, 27, 28, 29, 30, 31, 32, 33, 34 or 35%. In embodiments, the threshold may be within a range formed by selecting any two numbers (two percentages) listed in the immediately previous sentence (e.g., the NRPC may be selected if the expression rate for CD26 is at least above about 10%, between about 10% and about 20%, between about 15% and about 25%, etc.).

In some embodiments, T-NRPCs may be identified and selected based on the expression rate for CD141 being at least above a threshold, which may be about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%. In embodiments, the threshold may be within a range formed by selecting any two numbers (two percentages) listed in the immediately previous sentence (e.g., the NRPC may be selected if the expression rate for CD141 is at least above about 10%, between about 10% and about 20%, between about 15% and about 25%, etc.).

### NRPCs can be Selected Based on Expression Levels of CD26 and CD141 when the NRPC or NRPC-Candidates are in their Working Cell Bank Stage

In some embodiments, T-NRPCs can be selected based on expressions rates when the T-NRPCs or T-NRPC candidates are in a working cell bank (WCB) stage (e.g., immediately after thawing from a frozen state (e.g., before any subsequent passages after thawing)). In a WCB stage, T-NRPCs may be identified and selected when the expression rate for CD26 is under 5%. T-NRPCs may be identified and selected based on the expression rate for CD26 being at least below a threshold, which may be about 0.5%, 1%, 1.5%, 2%, 2.5%, 3%, 3.5%, 4%, 4.5%, 4.75%, 4.8%, 4.85%, 4.9%, 4.95% or 5%. In a WCB stage, T-NRPCs may be identified and selected when the expression rate for CD141 is under 30%. T-NRPCs may be identified and selected based on the expression rate for CD141 being at least below a threshold, which may be about 1%, 5%, 10%, 15%, 20%, 25%, 28%, 28.5%, 29%, 29.5%, or 30%. In embodiments, T-NRPCs may be identified and selected when the expression rate for CD26 is under 5% and when the expression rate for CD141 is under 30%.

In embodiments, T-NRPCs may be selected based on a threshold that is different from the above examples. In certain embodiments, T-NRPCs may be identified and selected based on the expression rate for CD26 being at least below a threshold, which may be about 5%, 7.5%, 10%, 12.5%, 15%, 17.5%, 20%, 22.5%, 25%, 27.5%, 30%, 32.5% or 35%, the expression rate for CD26 being measured when the T-NRPC or T-NRPC candidate is at the working cell bank stage. In embodiments, T-NRPCs may be identified and selected based on the expression rate for CD141 being at least below a threshold, which may be about 32.5%, 35%, 37.5%, 40%, 42.5%, 45%, 47.5%, or 50%, the expression rate for CD141 being measured when the T-NRPC or T-NRPC candidate is at the working cell bank stage.

### NRPCs can be Selected Based on Characteristic Expression Patterns of CD26 and CD141

Also or alternatively, the T-NRPCs can be identified based on the difference in expression rates of CD26 and CD141 by the NRPCs when compared to the expression rates of CD26 and CD141 by the respective MSCs. That is, the characteristic expression pattern by the desired NRPC (e.g., T-NRPC) may show a decreased expression rate when compared to its respective MSC from which the NRPC differentiated. For example, in the specific example shown in FIG. 3, the expression rate for CD26 decreased from 48.48% by T-MSCs to 21.77% by T-NRPCs (a decrease of 26.71%), and the expression rate for CD141 decreased from 60.36% by T-MSCs to 23.75% by T-NRPCs (a decrease of 36.61%). In contrast, the expression rate for CD26 decreased from 90.03% by AD-MSCs to 27.98% by AD-NRPCs (a decrease of 62.05%) while the expression rate for CD141 actually increased from 36.95% by AD-MSCs to 44.96% by AD-NRPCs (an increase of 8.01%). In some embodiments, T-NRPCs may be identified and selected based on the decrease (i.e., the delta) in CD26 expression rate between the CD26 expression rate by the NRPCs and the CD26 expression rate by the respective MSCs. The decrease in the CD26 expression rate may be at least above a threshold, which may be about 10%, 15%, 20%, 25%, 30%, 35%, 40%, 45%, or 50%. In embodiments, the threshold may be within a range formed by selecting any two numbers (two percentages) listed in the immediately previous sentence (e.g., the NRPC may be selected if the decrease in the CD26 expression rate between the CD26 expression rate by the NRPCs and the CD26 expression rate by the respective MSCs is at least about 10%, between about 10% and about 20%, between about 15% and about 25%, etc.). In some embodiments, T-NRPCs may be identified and selected based on the decrease (i.e., the delta) in CD141 expression rate between the CD141 expression rate by the NRPCs and the CD141 expression rate by the respective MSCs. The decrease in the CD141 expression rate may be at least above a threshold, which may be about 10%, 20%, 25%, 30%, 35%, 40%, 45%, 50%, or 55%. In embodiments, the threshold may be within a range formed by selecting any two numbers (two percentages) listed in the immediately previous sentence (e.g., the NRPC may be selected if the decrease in the CD141 expression rate between the CD141 expression rate by the NRPCs and the CD141 expression rate by the respective MSCs is at least above about 20%, between about 20% and about 30%, between about 25% and about 45%, etc.).

### T-NRPC was Found to Express Cytokines Related to Nerve Regeneration at a Significantly Higher Level than Other NRPCs

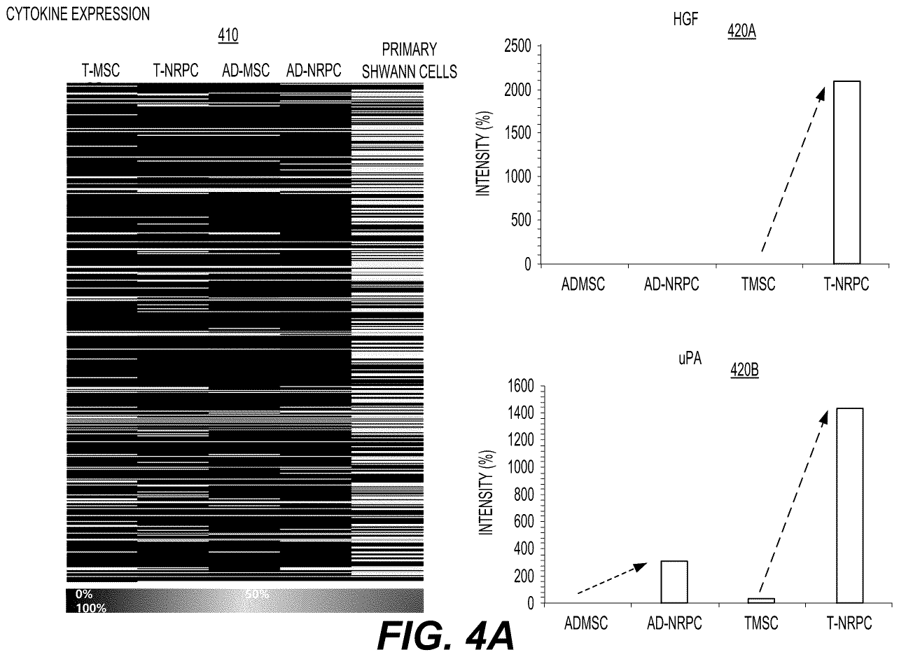

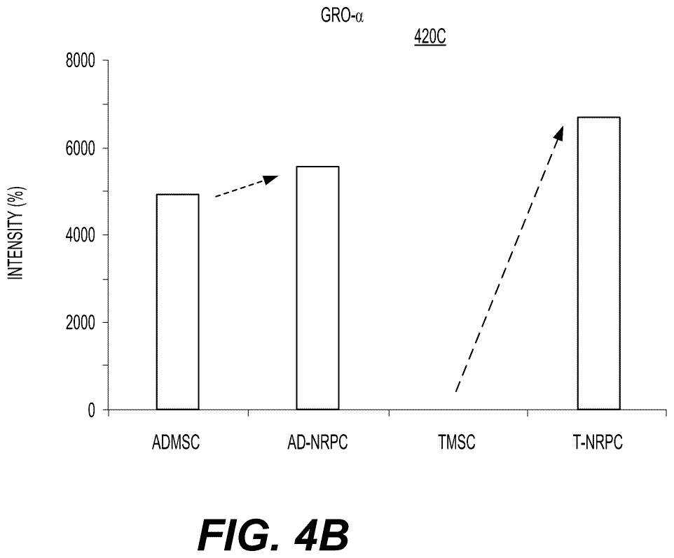

Cytokines are small proteins important in cell signaling. In particular, various cytokines are known to facilitate signaling between NRPCs and other cells for the treatment of damaged nerve cells. Such cytokines include: the hepatocyte growth factor (HGF), urokinase-type plasminogen activator (uPA), and growth regulated oncogene-alpha (GRO-α). In order to provide more effective NRPCs at treating damaged nerve cells, it would be useful to screen NRPCs for their ability to express such cytokines at sufficient expression rates. The inventors tested MSCs derived from various regions of a human (T-MSCs derived from tonsils and AD-MSCs derived from adipose tissue) as well as the respective NRPCs derived from the MSCs (T-MSCs and AD-MSCs) for their ability to sufficiently express such cytokines—HGF, uPA, and GRO-α. The expression rates were compared to a control group (shown in the heat map 410 of FIG. 4A as “Primary Schwann Cells”). FIG. 4A and FIG. 4B illustrate the cytokine expression rate among the MSCs and NRPCs derived from the various regions via a heat map 410, and graphs 420A-C, according to an example embodiment of the present disclosure. The inventors discovered that T-NRPC was found to express cytokines related to nerve regeneration at a significantly higher level than other NRPCs differentiated from MSCs from other regions of the human. For both the adipose tissue-derived and the tonsil derived sample groups (e.g., MSCs and NRPCs), the NRPC samples had greater expression of the aforementioned cytokines than the respective MSC samples from which the NRPC samples differentiated. For example, the T-NRPC samples were the only samples among the others (AD-MSC, AD-NRPC, and T-MSC samples) to express the HGF cytokine. Although the AD-NRPC samples expressed uPA at a greater rate (i.e., at 300%) than the AD-MSC samples, the T-NRPC samples expressed uPA at a far greater rate (i.e., a 1400%) than any of the remaining samples, including the T-MSC samples. Furthermore, although the AD-MSC sample expressed GRO-α (i.e., at an intensity of 5000%) whereas the T-MSC sample's expression rate for GRO-α was insignificant, T-NPC expressed GRO-α at a greater rate than any of the samples, at 7000%. Thus, based on the expression rates for cytokines relevant for nerve regeneration, the inventors discovered that T-NRPCS, as opposed to AD-NRPCs, was the more superior choice of NRPCs for the treatment of damaged nerve cells.

### NRPCs can be Used for Myelination and Improving the Stability of Nerves by Co-Culturing the NRPCs with Dorsal Root Ganglion (DRG) Neurons

The dorsal root ganglion (DRG) neurons are a cluster of neurons in the dorsal root of the spinal nerve. It has been found that when NRPCs are co-cultured with dorsal root ganglion (DRG) neurons, the NRPCs can be induced to transdifferentiate into Schwann cell-like cells that can myelinate (e.g., ensheath) the axons of DRG neurons. As previously discussed, myelination around axons insulates the axons and allow electrical signals to propagate through neurons more effectively over long distances. Thus, by facilitating myelination in DRG neurons, NPRCs can treat damaged nerve cells by myelinating damaged neurons and/or by forming new myelinated neurons.

### T-NRPCs can be Assessed for their Ability to Induce Myelination Based on MBP Expression in Dorsal Root Ganglia

Based on these experiments, it was discovered that NRPC candidates can be assessed for their ability to induce myelination by detecting expression of critical proteins such as MBP and/or precursors such as NF-H. For example, NRPCs and/or NRPC candidates can be co-cultured with dorsal root ganglia cells. The ability for the NRPCs and/or NRPC candidates to induce myelination can be assessed by subsequently examining the dorsal root ganglia and confirming myelination based on the expression of MBP proteins in the samples containing the dorsal root ganglia. In some embodiments, the expression of MBP proteins may be detected through immunofluorescence, for example, after performing immunofluorescence staining and checking for immunofluorescence sensitivity. Since the generation, development, and myelination of nerve fibers and other feeder cells from dorsal root ganglia are affected by cell culture conditions, co-culturing with dorsal root ganglia with the NRPC candidate samples may thus be used to assess the ability of each NRPC candidate sample to myelinate the feeder cells of the dorsal root ganglia. Although dorsal root ganglia is used in this experiment, since dorsal root ganglia is known, at least among human beings, to cause the generation, development, and myelination of nerve fibers and other feeder cells, it is contemplated that equivalent cells as the dorsal root ganglia found in other species can be used to evaluate abilities for NRPC candidate samples to induce myelination in the other species.

### NRPCs are Able to Promote Myelination when Co-Cultured with DRG Neurons More Effectively, as they Progress Through Stages of the NRPC Production

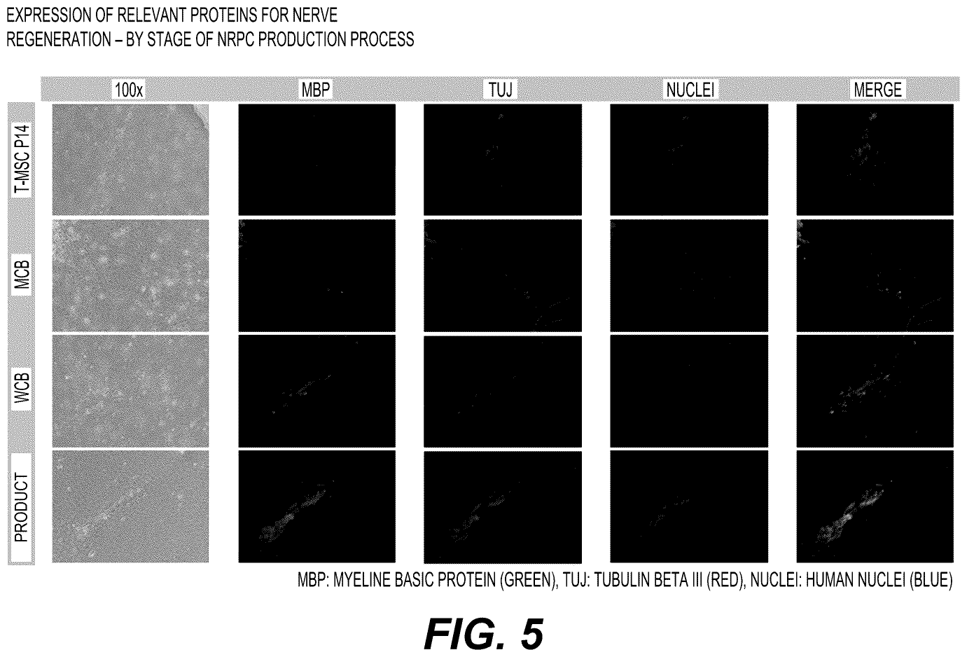

NRPCs are able to differentiate and myelinate axons of DRG neurons or otherwise improve the stability of DRG neurons through the expression of relevant proteins, such as MBP and TuJ1. In particular, MBP is important in the myelination process of nerves, while TuJ1 contributes to the stability of nerve cell bodies and microtubules. However, the inventors discovered that the expression rates for such proteins increase the further that NRPCs are in their production process. Specifically, the inventors obtained samples of NRPCs from three different stages of the NRPC production process (i.e., from the master cell bank (MCB), working cell bank (WCB), and product stages), and co-cultured the obtained samples with DRG neurons to observe the expression of proteins relevant for myelination and improved neuron stability. The inventors also obtained a sample of MSCs (from passage 14) to serve as a control. The rate of a protein expression was indicated by the intensity of fluorescence measured via a fluorescent marker on the NRPC samples. FIG. 5 is a set of immunofluorescence images showing the increased expression of proteins relevant for nerve regeneration for the NRPCs obtained from the three stages the NRPC production process, according to an example embodiment of the present disclosure. As shown in FIG. 5, the stages of the NRPC production process, from which samples of NRPCs were obtained, are the 14^{th} passage of a T-MSC culture (T-MSC P14) to serve as a control, a T-NRPC sample obtained from a master cell bank (MCB), a T-NRPC sample obtained from a working cell bank (WCB), and product. As used herein, the master cell bank of T-NRPCs comprise aliquots of a single pool of T-NRPC cells differentiated from the T-MSCs under defined conditions, dispensed into multiple containers, and stored under defined conditions. The MCB is used to derive the working cell banks (WCB) of the T-NRPCs. The T-NRPCs from the WCB are thus at a later stage in the production process in comparison to the T-NRPCs from the MCB. The product comprises T-NRPCs that are ready for use in the treatment of damaged cells, and are derived from the WCB, thus making the T-NRPCs of the product at a later stage in the production process in comparison to the T-NRPCs from the WCB. After co-culturing the T-NRPC samples from each stage with DRG neurons, and measuring the expression of various proteins through immunofluorescence, the inventors discovered that the expression of MBP and TuJ1 increased through each stage of the T-NRPC production process (i.e., the expression rates increased from T-MSC P14 to MCB, from MCB to WCB, and from WCB to Product. The ‘Nuclei’ column of FIG. 5, illustrates the results of nuclear staining of DRG neurons co-cultured with T-NRPC samples from each stage of the NRPC production process. As shown from the stained nuclei in the ‘Nuclei’ column, the number of nuclei, indicative DRG neurons, changes at each stage of the NRPC production process. The ‘Merge’ column of FIG. 5 visualizes the merged images of MBP and TuJ1 protein expression along with nuclear localization. By analyzing the information from the ‘Nuclei’ and ‘Merge’ columns, one can gain insights into the movement of cells involved in myelination. The increased expression of the MBP and TuJ proteins allowed cell communication to increase via myelination and neural tube formation to be induced.

### A Neurite Outgrowth Assay Involving Neuroblastoma Cells May be Used to Determine the Efficacy of NRPCs in Inducing Neurite Growth

A neurite (or neuronal process) projects from the cell body of a neuron and is known to be involved in the transport of the substances necessary for growth and regeneration of axons, neurotransmitters, nerve growth factors, etc. A neurite outgrowth assay can be conducted to compare neurite growth induced by neuronal regeneration-promoting cells, such as those described in the present disclosure. Certain clonal lines of mouse neuroblastoma cells, such as N1E-115 are known to extend or retract axons depending upon the culture media, can thus be used in the neurite outgrowth assay to test the ability of the presently disclosed NRPCs to induce neurite growth.

### The Neurite Outgrowth Assay Proved that T-NRPCs were More Successful at Inducing Neurite Growth than Other NRPCs

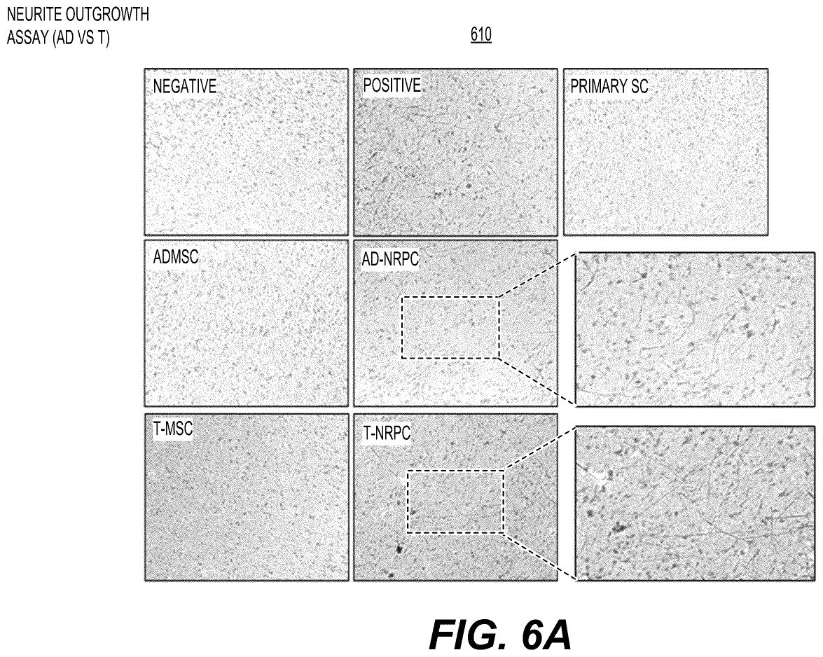

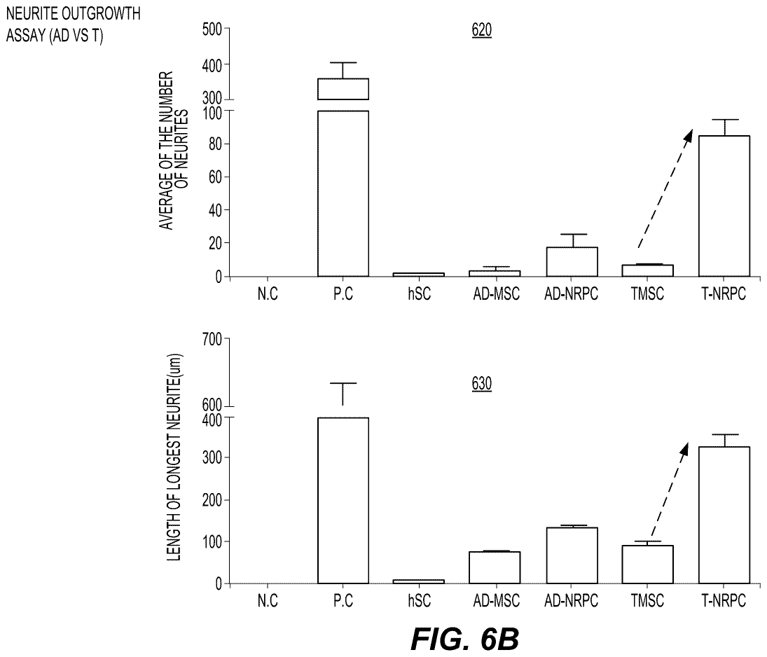

To test the efficacy of various NRPCs at inducing neurite growth, N1E-115 cells (mouse neuroblastoma cells, ATCC, USA) were cultured and seeded on a microporous filter (neurite outgrowth assay kit, Millipore, USA). The seeded cells were cultured for 48 hours in culture media from which the NRPC and MSC samples derived from various regions of the human were collected. The culture medium of each NRPC or MSC sample reflected active ingredients (e.g., proteins) expressed and/or produced by the respective NRPC or MSC sample. Absorbance was measured after staining the neurites projected through a fine porous filter. It was confirmed from the neurite outgrowth assay that the culture of the NRPCs regulates or stimulates the growth of neurites (axons) in the N1E-115 (mouse neuroblastoma) cells. However, the inventors discovered that different samples of NRPCs and MSCs stimulated neurite growth differently. FIG. 6A and FIG. 6B shows these different levels of neurite outgrowth among MSCs and NRPCs derived from various regions, using a set of images of the resulting culture 610 and graphs 620 and 630 quantifying the neurite growth. The different samples, for which results are shown, are of AD-MSCs, T-MSCs, AD-NRPCs, T-NRPCs, a negative control group, a positive control group, and a primary Schwann cell group. Graph 620 shows the average of the number of neurites among the cells of each sample group, while graph 630 shows the length of the longest neurite (in μm) of each sample group. The inventors discovered that the number of axons and the length of the longest axon increased significantly in the neurite outgrowth assay group that used the culture medium of the T-NRPC sample compared to the rest of the neurite outgrowth assay groups, which used culture media of the remaining samples (T-MSC, AD-MSC, and AD-NRPC samples). Thus, it was confirmed that the development of nerve cells, as demonstrated by the neurite outgrowth on the N1E-115 neuroblastoma cells caused by the culture medium of the T-NRPC sample, can be greatly enhanced by T-NRPCs.

### There is Variation in the Ability to Induce Neurite Outgrowth Even Among Tonsil-Derived NRPC and MSC Samples

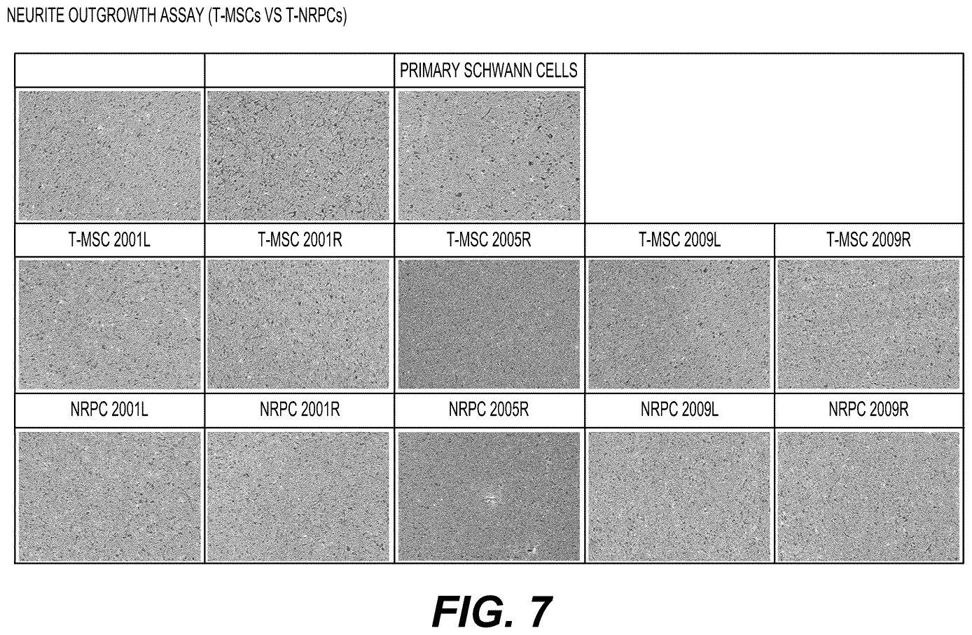

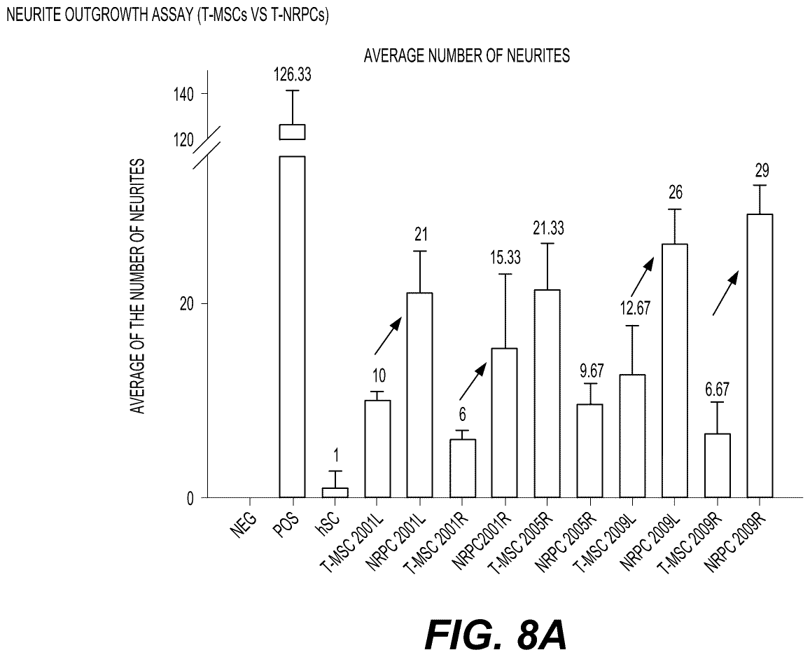

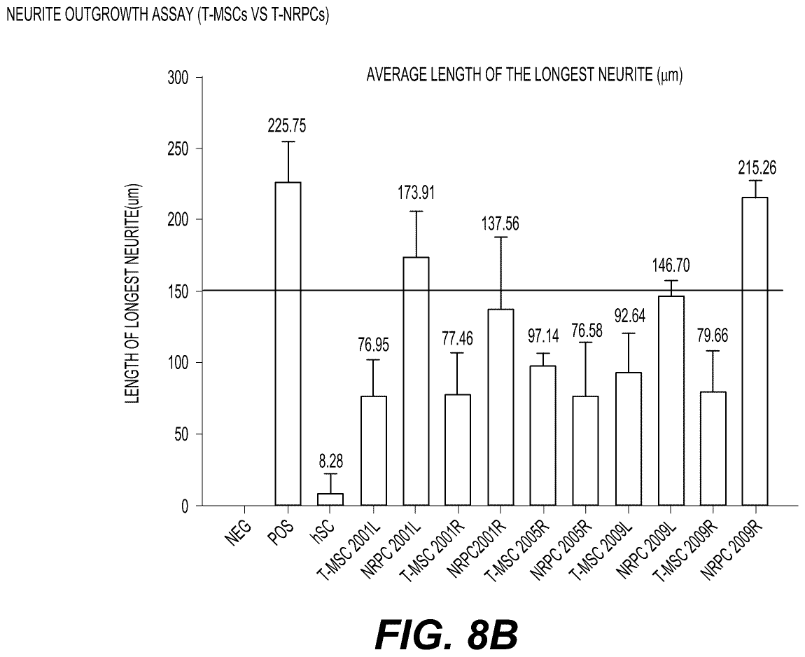

Neurite outgrowth assays were also performed using culture media from different samples of tonsil-derived MSCs (T-MSCs) and tonsil-derived NRPCs (T-NRPCs). The different samples corresponded to MSCs obtained from different humans (indicated in the sample name by an identification of the human (e.g., given by numbers such as 2001, 2005, etc.)), and/or from different sides of the tonsil (indicated in the sample name by “L” for left tonsil and “R” for right tonsil). To test the efficacy of the culture media of these various T-NRPC and T-MSC samples at inducing neurite growth, N1E-115 cells (mouse neuroblastoma cells, ATCC, USA) were cultured and seeded on a microporous filter (neurite outgrowth assay kit, Millipore, USA). The seeded cells were cultured for 48 hours in culture media from which the different T-NRPC and T-MSC samples derived from different humans or sides of the tonsils. Absorbance was measured after staining the neurites projected through a fine porous filter. The results of this experiment are illustrated in FIGS. 7, 8A and 8B. Specifically, FIG. 7 is a set of images showing the different neurite outgrowth among samples of tonsil-derived MSCs (T-MSCs) and tonsil-derived NRPCs (T-NRPCs). FIG. 8A and FIG. 8B present a set of graphs quantifying the neurite outgrowth based on the average number of neurites and average length of the longest neurites among the neurite growth assay groups corresponding to culture media of the different samples of tonsil-derived MSCs (T-MSCs) and tonsil-derived NRPCs (T-NRPCs). Moreover, each T-NRPC sample may correspond with a respective T-MSC sample from which it is derived, with each pair of samples (a T-NRPC sample and its respective T-MSC sample) corresponding to an individual human (identifiable by numbers such as 2001, 2005, 2009, etc.) and tonsil side (identifiable by ‘L’ for left or ‘R’ for right in the sample names). The results shown in FIGS. 7, 8A and 8B confirm that, as the T-MSC samples differentiated into their respective T-NRPC samples, there was generally an increase in neurite outgrowth in the N1E 115 cells induced by the culture medium of the sample. Thus, the culture medium of T-NRPC 2001L induced greater neurite outgrowth than the culture medium of T-MSC 2001L; the culture medium of T-NRPC 2001R induced greater neurite outgrowth than the culture medium of T-MSC 2001R; the culture medium of T-NRPC 2009L induced greater neurite outgrowth than the culture medium of T-MSC 2009L; and the culture medium of T-NRPC 2009R induced greater neurite outgrowth than the culture medium of T-MSC 2009R. There was an increase in neurite outgrowth as measured by both an increase in the average number of neurites in the induced N1E 115 cell sample and as measured by the length of the longest neurite in the induced N1E 115 cell sample. There was an outlier, however, with respect to T-MSC2005R and T-NRPC2005R. Based on the results of the neurite outgrowth assay shown in FIGS. 7, 8A and 8B, the inventors discovered that, while T-NRPC generally has the ability to effect nerve regeneration (e.g., through neurite outgrowth), it is nevertheless crucial to further screen T-NRPC samples further (e.g., based on human and tonsil side from which the T-NRPCs are derived via their corresponding T-MSC) to select and expand the most effective T-NRPCs.

### NRPCs can be Further Selected Based on their Ability to Induce Neurite Outgrowth as Determined by Meeting Thresholds for the Average Number of Neurites Formed

In particular, the inventors discovered NRPCs can be screened, selected, and expanded based on their ability to induce neurite outgrowth (e.g., in neuroblastoma cells), and a criteria may be established for assessing which NRPCs induce neurite outgrowth most effectively. In an embodiment, a given NRPC is able to induce neurite outgrowth if an average number of neurites formed per neuroblastoma cell in the respective sample of neuroblastoma cells is at least 15, and a length of a longest neurite formed in the respective sample of neuroblastoma cells is at least 145 μm. However, in some embodiments, the threshold for the number of neurites formed per neuroblastoma cell, to determine the ability of an NRPC sample to induce neurite growth, may be higher. For example, the threshold for the number of neurites formed per neuroblastoma cell may be about 15, 16, 17, 18, 19, 20, 21, 22, 23, 24, 25, 26, 27, 28, 29, 30, 40, 50, or 100. In embodiments, the threshold may be within a range formed by selecting any two numbers listed in the immediately previous sentence (e.g., the minimum threshold number of neurites formed per neuroblastoma cell may be about 12, between about 10 and about 30, etc.). In some aspects, the average number of neurites formed per sample may be determined by averaging the number of neurites formed among a plurality of samples of neuroblastoma cells. Each sample may be contained within a respective well. For example, the number of neuroblastoma cells (e.g., N1E-115 mouse neuroblastoma cells) used in the neurite outgrowth assay may be 1×10⁶ cells/ml or 1×10⁵ cells/well). In some aspects, a plurality of samples (wells) may be used. In one embodiment, 20-40 samples (wells) of N1E-115 mouse neuroblastoma cells may be used, for example, 24 samples via a plate containing 24 wells. NRPCs can be assessed for their ability to induce neurite growth by measuring the number of neurites formed per neuroblastoma cell per sample to obtain the average.

### NRPCs can be Further Selected Based on their Ability to Induce Neurite Outgrowth as Determined by Meeting Thresholds for the Average Length of the Longest Neurite Formed

As previously discussed, NRPCs can be screened, selected, and expanded based on their ability to induce neurite outgrowth (e.g., in neuroblastoma cells), and a criteria may be established for assessing which NRPCs induce neurite outgrowth most effectively. In an embodiment, a given NRPC sample is able to induce neurite outgrowth if an average length of a longest neurite formed in the respective sample of neuroblastoma cells is at least 145 μm. In some embodiments, the threshold for the length of the longest neurite formed in the sample of neuroblastoma cells (to determine the ability of an NRPC sample to induce neurite growth in the sample of neuroblastoma cells) may be higher. For example, the threshold for the length of the longest neurite formed in the sample of neuroblastoma cells may be about 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, or 225 μm. In embodiments, the threshold may be within a range formed by selecting any two numbers listed in the immediately previous sentence (e.g., the minimum threshold for the average length of the longest neurite formed in the sample of neuroblastoma cells may be about 150 μm, between about 150 μm and about 170 μm, etc.). In some aspects, the average length of the longest neurite formed may be determined by averaging the longest length of neurites formed among a plurality of samples of neuroblastoma cells. In some aspects, a plurality of samples (e.g., 20-40 samples of neuroblastoma cells may be used). For example, 24 samples (contained in respective wells) may be used via a 24 well plate. Furthermore, 1×10⁵ N1E 115 mouse neuroblastoma cells per well may be seeded. The NRPCs can be assessed for their ability to induce neurite growth (e.g., by measuring the length of the longest neurite formed) in each sample (well).

### Neurite Outgrowth Among Samples Involving Tonsil Derived MSCs and NRPCs Decreases when the Expression of CD121a is Reduced

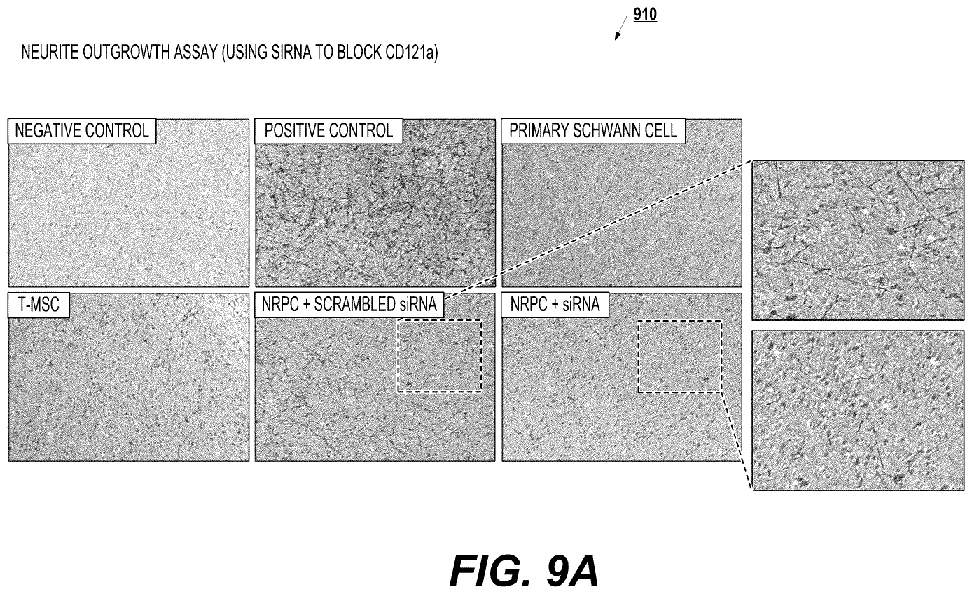

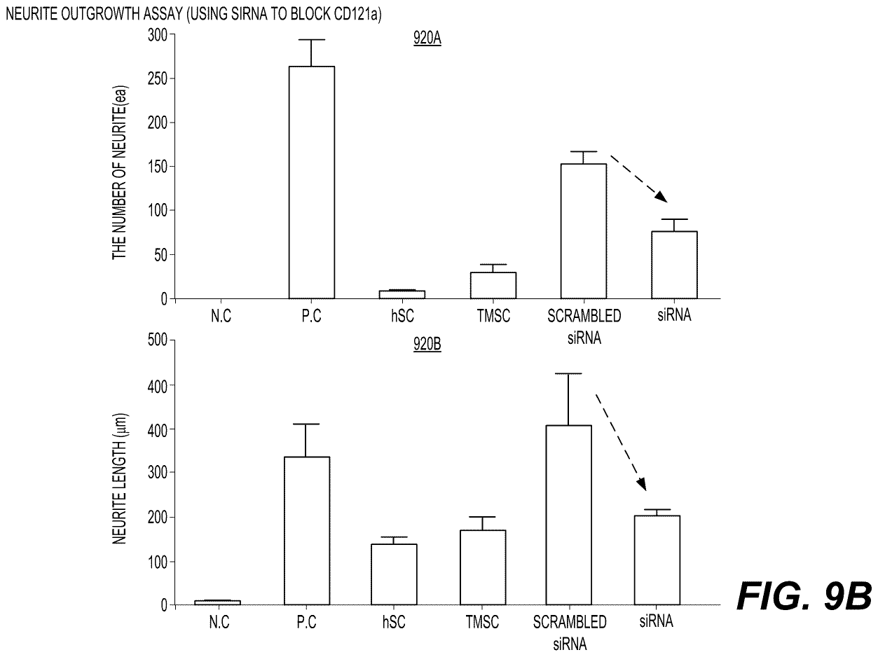

As previously discussed, neurite outgrowth in a precursor or damaged nerve cell (e.g., mouse neuroblastoma N1E 115 cell line) may occur more effectively under certain culture media (e.g., those where T-NRPCs are cultured) over other culture media based on the active ingredients of such culture media. Such active ingredients may be proteins expressed by NRPCs grown in the culture media that are influential in inducing neurite outgrowth. The inventors have discovered CD121a as a particularly important protein for neurite outgrowth, after performing tests that showed that neurite outgrowth decreased when CD121a expression was reduced. In these tests, small interfering RNAs (siRNAs) were used to reduce the expression of the CD-121a. Specifically, the tests involved six sample groups—a negative control group, a positive control group, a primary Schwann cell sample, T-MSC sample, a T-NRPC sample treated with a scrambled form of the siRNA, and a T-NRPC sample treated with the siRNA. The scrambled siRNA served as a negative control, distinguishing specific gene effects from non-specific or cell-related effects. Furthermore, the scrambled siRNA served to reduce non-specific effects and background noise, thus improving the accuracy and reliability of the experiment. FIGS. 9A and 9B include a set of images 910 showing the results in these sample groups, and graphs 920A and 920B showing the number of neurites formed and the length of the longest neurite formed among the sample groups. As shown in FIGS. 9A and 9B, neurite growth decreases significantly among samples of T-MSCs and T-NRPCs when the expression of the CD121a was reduced using the small interfering RNA (siRNA). On the other hand, the scrambled siRNA functioned to exclude the effect of siRNA transfection on cells when knocking down the target gene. Thus, in the sample group involving T-NRPCs treated with scrambled siRNA, the number of neurites was observed more than in the other experimental groups, and the length of axons was longer than that of the positive control. Thus, the inventors discovered that CD121a marker, which was most highly expressed in T-NRPCs, is an important marker for nerve regeneration and, and the number of axons and the length of the longest axon decreased when CD121a expression was reduced through a knock down of CD121a.

### CD121a Expression Varies Among Different Samples of MSCs and NRPCs and Across NRPC Production Stages

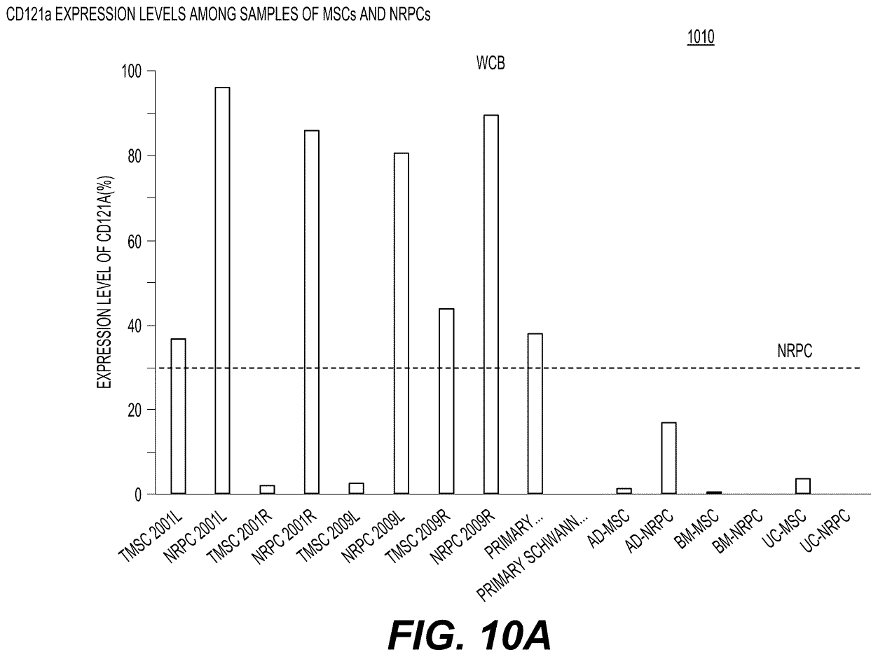

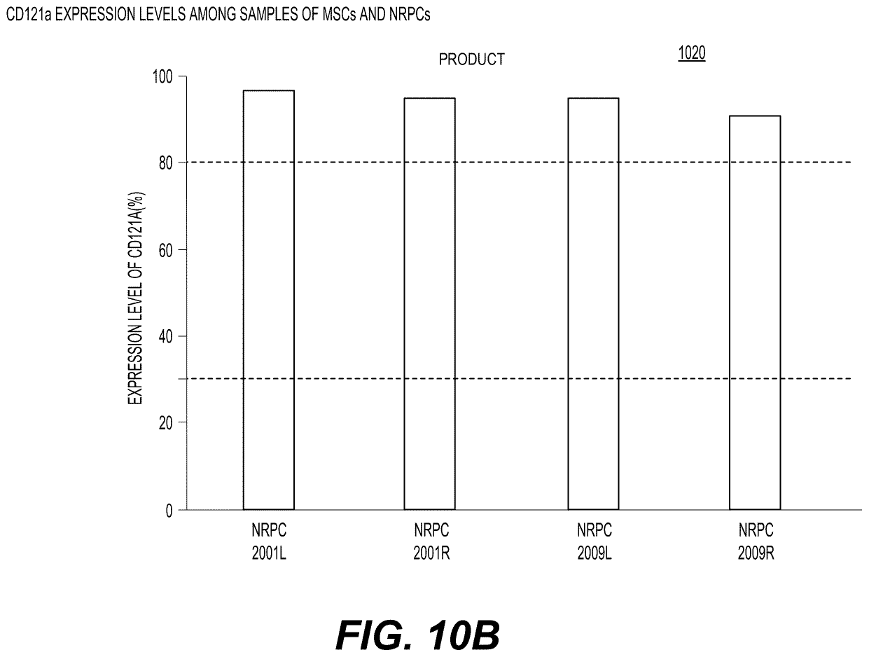

Having established that CD121a is critical for effective neurite outgrowth, the inventors investigated how CD121a expression varies among different kinds MSCs and NRPCs, such as MSCs and NRPCs derived from different humans, different regions of a human (including tonsil sides), or obtained from different stages of the NRPC production process. The inventors obtained several sample groups involving various TMSCs, MSCs, primary Schwann cells, and control groups from working cell banks to test for expression of CD121a—TMSC 2001L, NRPC 2001L, TMSC 2001R, NRPC 2001R, TMSC 2009L, NRPC 2009L, primary Schwann cells, AD-MSC, AD-NRPC, BM-MSC, BM-NRPC, UC-MSC, UC-NRPC. Of these samples, which were obtained from the working cell bank stage of the production process, the inventors were able to determine which of the samples continued to express CD121a at significant levels (e.g., above 75%) in the product stage. The inventors obtained these select T-NRPC samples from the product stage of the production process to investigate how CD121a expression increases through the production process. These select T-NRPC samples—T-NRPC 2001L, T-NRPC 2001R, T-NRPC 2009L, and T-NRPC 2009R—corresponded to T-NRPCs derived from different humans (the humans identifiable by numbers in the sample name, such as 2001, 2005, 2009, etc.) and/or different tonsil sides (as indicated by ‘L’ for left or ‘R’ for right in the sample name). The results pertaining to CD121a expression levels are shown in FIGS. 10A and 10B. Specifically, graph 1010 of FIG. 10A shows the expression levels of the marker CD121a among samples of MSCs and NRPCs derived from various regions and humans and obtained from working cell banks, while graph 1020 of FIG. 10B shows the expression levels of CD121a among (final) products for sample groups of T-NRPC 2001L, T-NRPC 2001R, T-NRPC 2009L, and T-NRPC 2009R.

### By Screening for and Selecting NRPC Candidates with CD121a Expression Levels of at Least 30%, the Quality of NRPCs Improves Significantly

Based on the CD121a expression levels shown in FIGS. 10A and 10B, the inventors discovered that more effective NRPCs (e.g., based on the potential for improved neurite outgrowth) can be produced by screening for NRPC candidates that have high level of CD121a expression. In particular, the inventors noted that, at an earlier stage of the NRPC production process (e.g., as MSCs differentiate into their respective NRPCs based on GMP standards), NRPC candidates in working cell bank (WCB) in which CD121a are expressed at more than 30% expression rates can be selected. For example, as shown in graph 1010, the NRPC candidate groups from the working cell bank (WCB) having a CD121a expression level at or higher than a first expression rate threshold (i.e., of 30%)—T-NRPC 2001L, T-NRPC 2001R, T-NRPC 2009L, and T-NRPC 2009R—were ultimately the T-NRPC samples that were able to express CD121a at levels above a higher second threshold expression rate (e.g., 80%) when these samples were in their product stage. Moreover, these samples were able to maintain a sufficient expression rate (at least 30%) at least while they were in a frozen state in the product stage. The expression rate was recovered to more than 80% expression level when these samples in their product stage where these samples were in an unfrozen state (e.g., one or more passages after thawing) and/or on a live standard. Thus, CD121a expression levels of 30% or more among NRPC candidate samples in the working cell bank (WCB) stage can be used as a criteria for determining which of these NRPC-candidates are to be selected to be used for the rest of the production process in forming the product of NRPCs. However, in some embodiments, the threshold for CD121a expression levels at the working cell bank stage (e.g., first threshold) or at the product stage (e.g., second threshold) may be higher. For example, from the working cell bank stage, NRPC-candidates may be selected if their CD121a expression levels are at least above a threshold, which is about 30%, 32.5, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, or 80%. In embodiments, the threshold may be within a range formed by selecting any two numbers (two percentages) listed in the immediately previous sentence (e.g., an NRPC candidate sample may be selected if the CD121a expression rate that is at least above a threshold, where the threshold is about 30%, between about 30% and about 80%, between about 35% and about 50%, etc.).

### Frozen Versus Unfrozen State

As discussed above, expression levels for CD121a appeared to increase significantly when measured one or more passages after the NRPCs and/or NRPC candidates were thawed from a frozen state compared to when the NRPCs and/or NRPC candidates were in a frozen state. A frozen state may refer to NRPCs and/or NRPC candidates being cryopreserved in a suitable medium (e.g., liquid nitrogen) in which the temperature is below a threshold, which is about −200° C., −195° C., −190° C., −185° C., −180° C., −175° C., −170° C., −165° C., or −160° C. In embodiments, the threshold may be within a range formed by selecting any two numbers (temperatures) listed in the immediately previous sentence (e.g., NRPCs and/or NRPC candidates may be in a frozen state if they are cryopreserved in a suitable medium at a temperature below a threshold, where the threshold is about −180° C., between about −190° C. and about −170° C., between about −180° C. and about −160° C., etc.). Expression rates or levels for protein markers in NRPCs or NRPC candidates in a frozen state may be determined by measuring the expression rate or level immediately after the NRPCs or NRPC candidates are thawed. By measuring the expression level at a time immediately after thawing, heat-dependent cell activities (e.g., enzymatic activities) that could affect the expression rate can be avoided. For example, the time after thawing considered to be immediately after thawing can be less than about 0.5 seconds, 1, seconds, 5 seconds, 30 second, 1 minute, 5 minutes, 10 minutes, 30 minutes, or 1 hour. In embodiments, the time after thawing that forms the upper limit for what is considered immediately after thawing may be within a range formed by selecting any two numbers (two times) listed in the immediately previous sentence (e.g., the time after thawing that forms the upper limit for what is considered immediately after thawing may be within 1 minute, between about 30 seconds to about 30 minutes, within 1 hour, etc.). In at least one embodiment, a characteristic of cells immediately after thawing (e.g., the expression level of a given marker protein in NRPCs or NRPC candidates immediately after thawing) may mean the characteristic of the cells before any subsequent one or more passages (e.g., subculturing) after thawing. NRPCs and/or NRPC candidates may be said to exist in a “live” state when they are at one or more passages after being thawed from a previously frozen state. It is contemplated that cells in the master cell bank (MCB) and working cell bank (WCB) stages of the NRPC production process are in the frozen state unless indicated otherwise. Furthermore, it is contemplated that cells (e.g., T-NRPCs) from the product (F/P) stage are in a live state unless indicated otherwise.

### Thawing Process for Cells

As previously discussed, in order to measure a characteristic (e.g., an expression level of a given marker protein (e.g., CD121a) from cells that are in a frozen state (e.g., in their working cell bank stage), the measurement may need to occur immediately after thawing (e.g., before any subsequent one or more passages after thawing). Thus, the measurement of the characteristic for cells in a frozen state may involve thawing the cells. Cells in a frozen state (e.g., NRPCs or NRPC candidates in their working cell bank stage) may be thawed by placing the cells in an environment having a predefined thawing temperature for a predetermined thawing period. The thawing temperature may be about 25° C., 30° C., 32.5° C., 35° C., 36° C., 37° C., 38° C., 39° C., 40° C., 42.5° C., 45° C., or 50° C. In some embodiments, may be within a range formed by selecting any two numbers (two temperatures) listed in the immediately previous sentence (e.g., the thawing temperature may be between about 36° C. and 38° C., between about 30° C. and 45° C. seconds, etc.). The thawing period may be about 2.5, 2.6, 2.7, 2.8, 2.9, 3, 3.1, 3.2, 3.3, 3.4, 3.5, 4, 4.5, or 5 minutes. In some embodiments, the thawing period may be within a range formed by selecting any two numbers (two times) listed in the immediately previous sentence (e.g., the thawing period may be between about 2.5 minutes and about 3.5 minutes, between about 3 minutes and about 4 minutes, etc.). In at least one embodiment, the thawing temperature is 37° C. and the thawing period is 3 minutes.

### CD121a Expression Levels for T-NRPCs Across Passages of the Product Stage are Consistently High

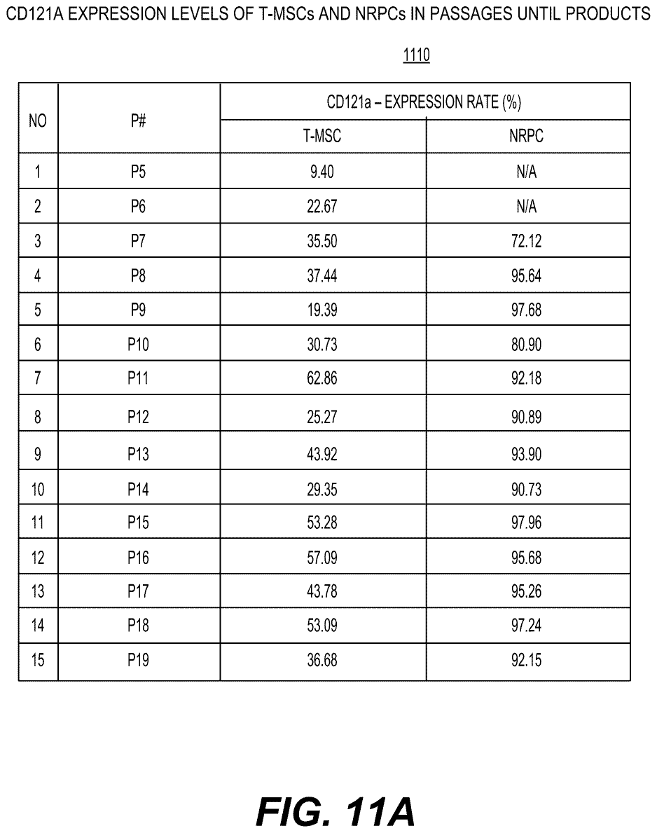

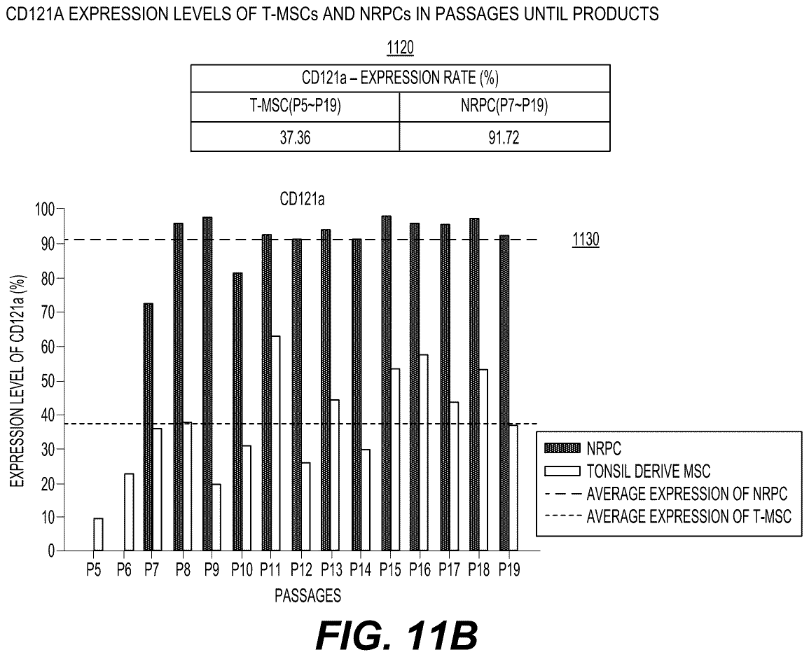

Having established that the product of appropriately selected NRPCs (e.g., T-NRPCs) are able to express CD121a to significant levels (e.g., at least 80%), the inventors investigated how the expression level of CD121a progress through various passages in the product stage of the NRPC production process. The inventors obtained sub-samples of T-MSC and NRPC at each passage from passages 5 through 19, and tested the sub-samples for expression of CD121a. FIGS. 11A and 11B shows the expression levels of CD121a of T-MSCs and NRPCs in passages until the products in table 1110, the average expression levels for T-MSC and T-NRPC each for passages 5 through 19 in table 1120, and a graph illustrating the expression levels of CD121a of T-MSCs and NRPCs in passages until the products. As shown in FIGS. 11A and 11B, the high CD121a expression characteristics of T-NRPC (e.g., of 80% or more in the live state) are continuously maintained until passage 19 even after the product. However, CD121a expression in the T-MSC sample varies across passages and the expression levels are typically lower.

### The CD121a Expression Levels Correlate with Neurite Outgrowth in Final Passages of T-MSCs and T-NRPC

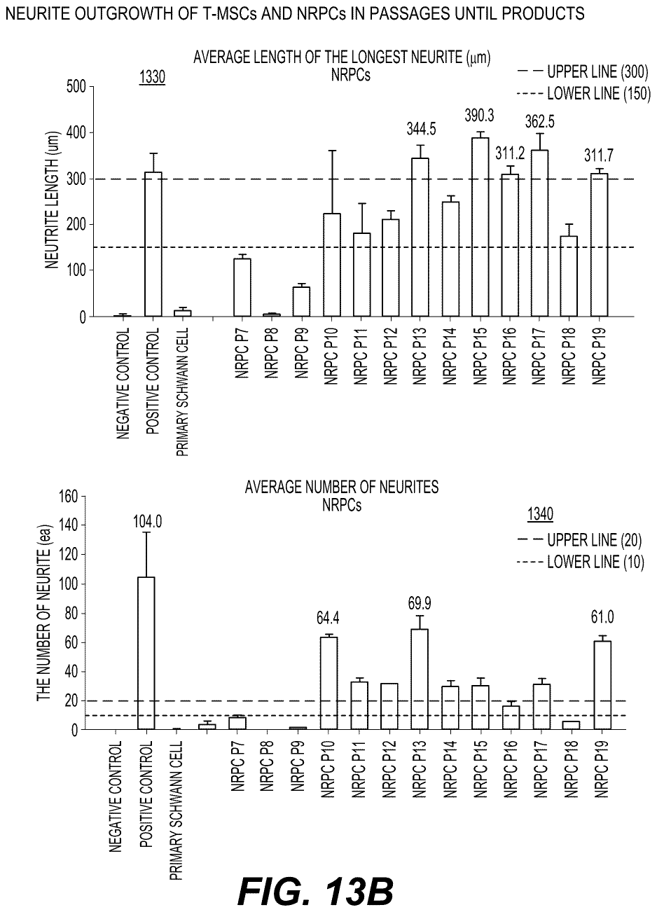

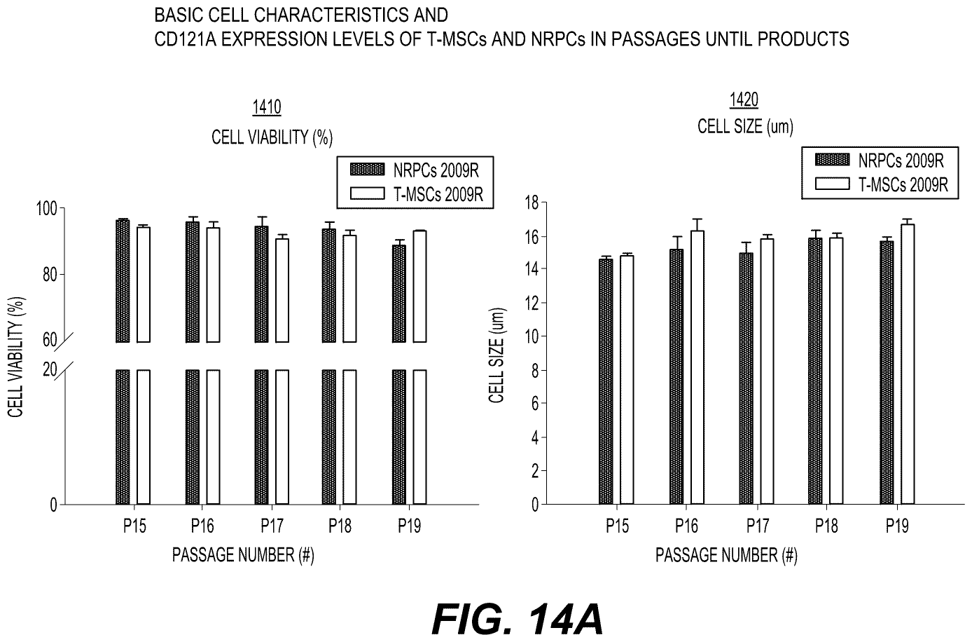

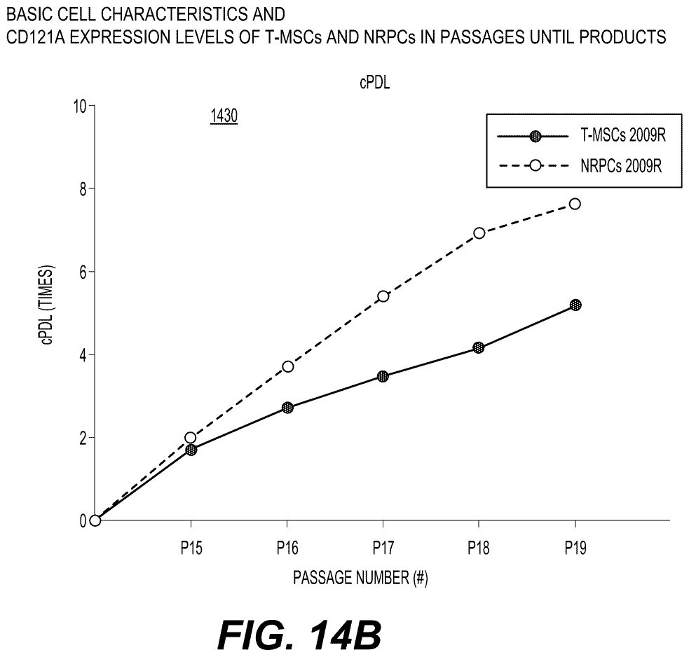

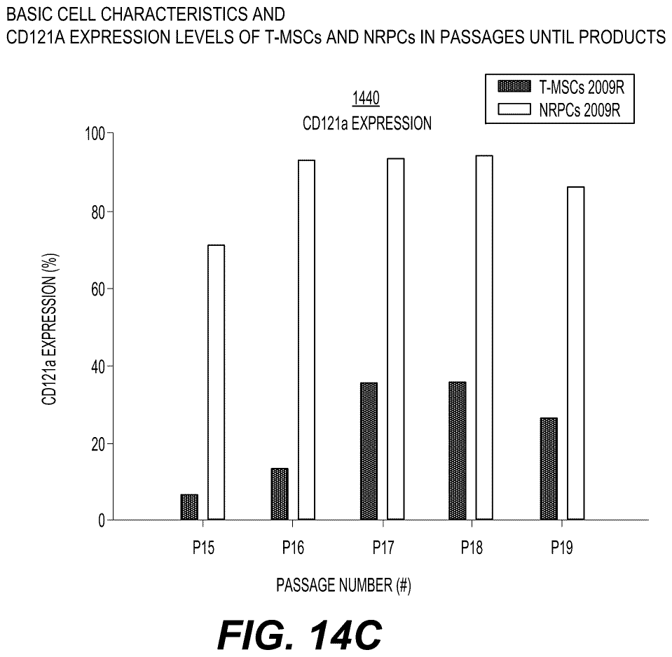

The inventors also observed that the consistently high CD121a expression levels across passages of the products of T-NRPCs coincided with consistently high neurite outgrowth in neurite outgrowth assays prepared using samples of the products of T-NRPCs obtained across passages. The inventors also observed that the varied CD121a expression levels across passages of the products of T-MSCs coincided with varied neurite outgrowth in neurite outgrowth assays prepared using samples of the products of T-MSCs obtained across passages. The results are shown in FIG. 12. Specifically, FIG. 12 includes a set of images 1210 showing the results of neurite outgrowth using samples until products of T-NRPCs and T-MSCs obtained across passages P14 through P19. In some embodiments, there is a correlation between neurite outgrowth assay and expression levels of the marker CD121a of T-MSCs and NRPCs in each passage until the products, as will be described in relation to FIGS. 14A to 14C. Furthermore, the inventors assessed the state of the cells by determining their cell viability and cell size.

### T-NRPCs Obtained Across Passages in the Product Stage are Able to Induce High Neurite Outgrowth

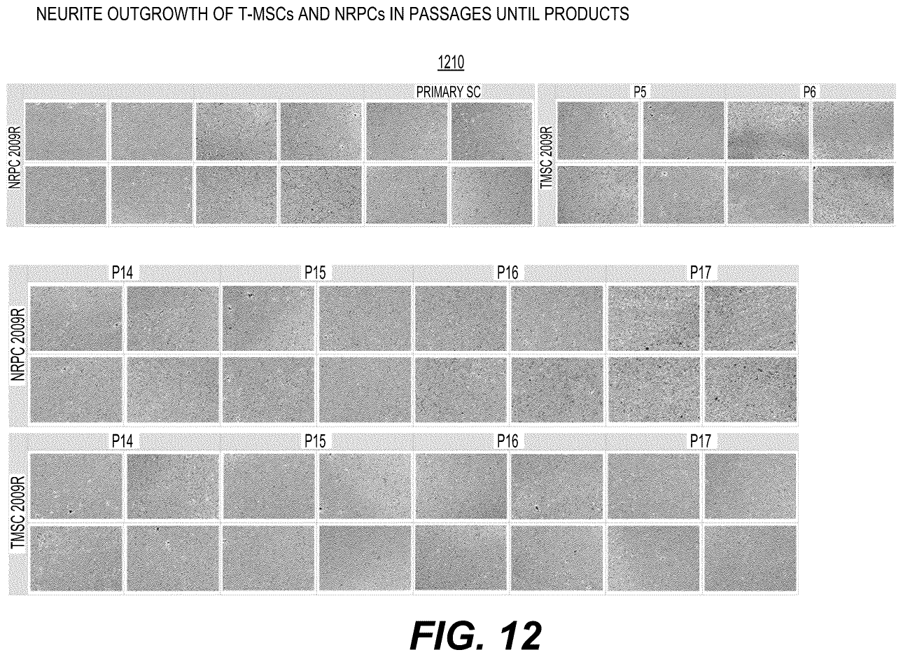

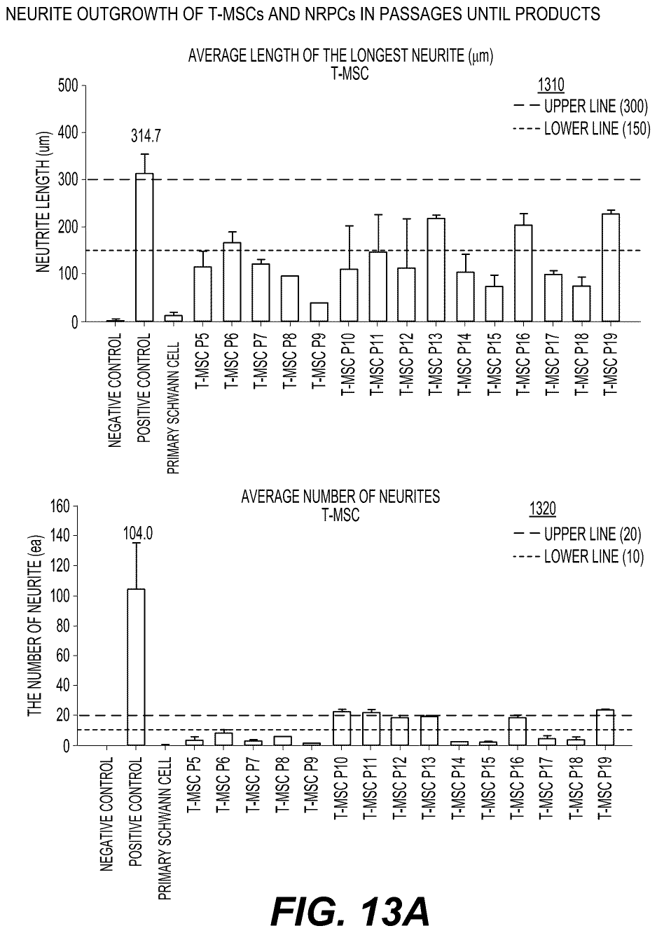

As previously discussed, T-NRPCs samples at the product stage are able to express CD121a at consistently high levels, which correlates with high neurite growth. The inventors have demonstrated, using neurite outgrowth assays involving T-MSCs and T-NRPCs obtained across passages of the product stage, that high neurite outgrowth is maintained. For example, FIGS. 13A and 13B indicate the neurite outgrowth observed in neurite growth assays involving product samples of T-MSCs and T-NRPCs obtained across passages. Here, the neurite outgrowth is indicated based on the number of axons and the length of the longest axon in each sample. Specifically, graph 1310 indicates the longest length of a neurite and graph 1320 indicates the average number of neurites observed in samples based on T-MSCs obtained across passages. Graph 1330 indicates the longest length of a neurite and graph 1340 indicates the average number of neurites observed in samples based on T-NRPCs obtained across passages in the product stage (e.g., after the MSCs have fully differentiated into T-NRPCs). For the samples based on T-MSCs obtained across passages, the number of axons and the length of the longest axon appear to vary. However, for samples based on T-MSCs obtained across passages in the product stage, the length of the longest axon and the number of axons and are maintained above thresholds even as far as passage 19 after NRPC differentiation. Specifically, as shown in graph 1330, the length of the longest axon in samples based on the product of T-NRPCs appears to be maintained above a lower threshold of 150 μm even as late as passage 19. Some samples based on the product of T-NRPCs even exceed a higher threshold of 300 μm. Furthermore, as shown in graph 1340, the average number of axons in samples based on the product of T-NRPCs appears to be maintained above a lower threshold of 10 even as late as passage 19. Some samples based on the product of T-NRPCs even exceed a higher threshold of 20.

### Products of T-NRPCs were Even More Effective at Expressing CD121a after Thawing

As previously discussed, the inventors discovered that CD121a is critical to significantly improving nerve regeneration, and that T-NRPCs are known to express the CD121a protein particularly well in their product stage. The inventors also discovered that CD121a expression levels significantly increase in the product stages, when compared to earlier stages (e.g., working cell bank stages). Expression rates for CD121a for the products of T-NRPCs were measured after the product samples were thawed and subcultured (e.g., after a plurality of passages). Specifically, in this study, the inventors measured the CD121a expression conducted on samples of products of T-NRPCs obtained across passages 15 through 19 after the samples were thawed (from a frozen state). For comparison, the corresponding T-MSC samples, from which differentiation to the T-NRPCs had occurred, were also obtained and tested for CD121a expression. For consistency, the T-NRPC and T-MSC samples belonged to the human and tonsil side corresponding to 2009R (i.e., the samples were T-NRPC 2009R and T-MSC 2009R). Additionally, to ensure that T-MSCs and T-NRPCs were viable for use in treatment after being thawed, the inventors also tested the resulting samples after being thawed for cell characteristics (e.g., cell viability, cell size, and cell population doubling levels (cPDL)). FIGS. 14A to 14C show the results of these tests. Specifically, graphs 1410, 1420 and 1430 show cell viability, cell size, and cell population doubling levels (cPDL)), respectively, of the product samples of T-NRPCs 2009R, compared to their corresponding T-MSCs 2009R samples, obtained across pages P15 through P19. Furthermore, graph 1440 shows the expression of the marker CD121a among the product samples of T-NRPCs 2009R, compared to their corresponding T-MSC 2009R samples, across passages 15 through 19. As shown be these graphs, the T-MSC and T-NRPC cell samples maintained healthy basic cell characteristics through each passage. Furthermore, the T-NRPC samples maintained high CD121a expression after being thawed. Specifically, an expression level of 70% was observed immediately after thawing (which indicated the likely expression level of the product at the frozen state), but the expression level recovered to more than 80% from passage 16 onwards (indicating the expression level at live, nonfrozen states). As previously discussed, the expression levels of CD121a among the T-MSC samples remained lower.

### Methods of Generating the Improved NRPCs Disclosed Herein Involve the Ability of MSCs to Differentiate into NRPCs and the Ability of the NRPCs to Express CD121a

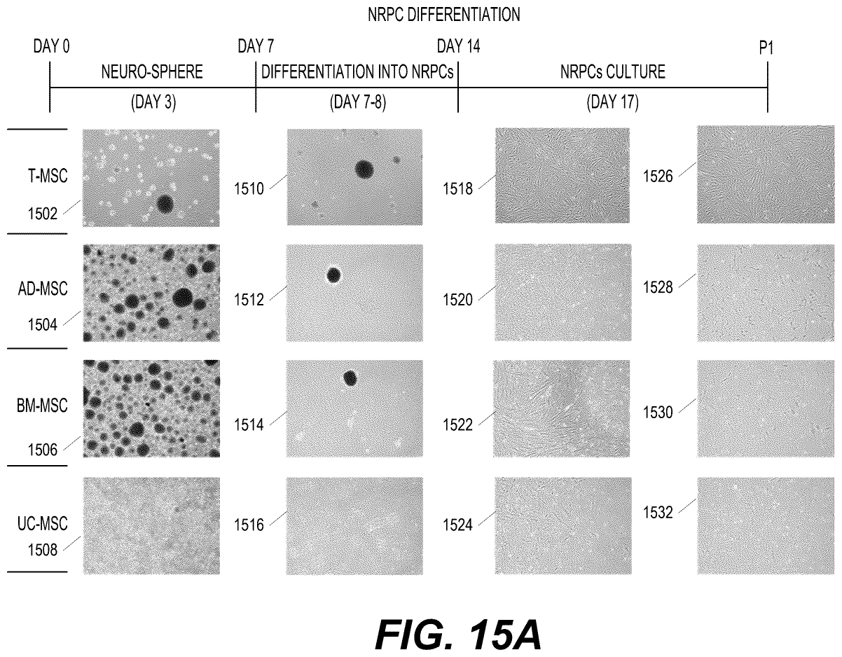

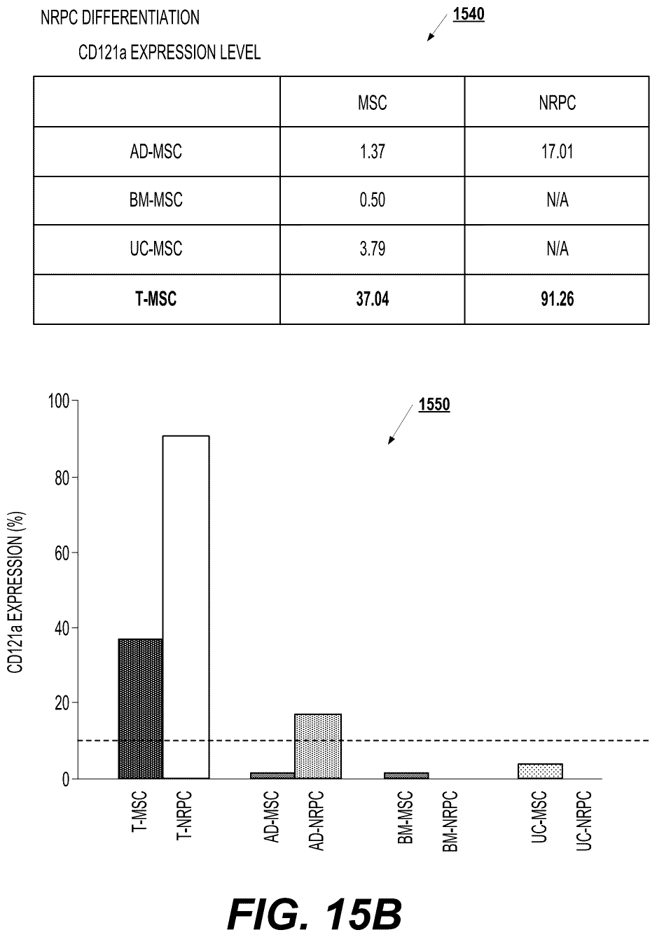

Thus, the present disclosure describes improved NRPCs and methods of generating them for the treatment and/or regeneration of damaged nerves. As described through the aforementioned experiments and tests, various embodiments of methods of generating the improved NRPCs involve using NRPCs differentiated from MSCs obtained from the tonsil of a human that express the protein markers CD26, CD106, CD112, CD121a, and CD141, selectively expanding such T-NRPCs candidates expressing the protein marker CD121a above threshold levels (e.g., above 30%), and selectively expanding T-NRPC candidates that exhibit neurite formation. For such embodiments, the ability of MSCs to be able to differentiate into NRPCs is significant. FIG. 15A shows a set of images 1502-1532 illustrating the differentiation of MSCs, derived from various regions, to NRPCs, according to an example embodiment of the present disclosure. The set of images show that in the process of differentiating into NRPCs, the MSCs form neurospheres or free flowing clusters of neural stem cells. The set of images 1502-1532 confirms that some MSCs are better able to differentiate to NRPC. For example, AD-MSC and T-MSC differentiated well into NRPC, whereas BM-MSC and UC-MSC did not differentiate as well into NRPC. Furthermore, as previously discussed, the ability of the MSCs and NRPCs to be able to express the critical CD121a protein also varies. FIG. 15B shows a set of graphs, 1540 and 1550, indicating the expression rate of the marker CD121a among NRPCs and their corresponding MSCs derived from various regions of human(s). The differences in expression levels of CD121a confirms that, while AD-MSC and T-MSC are better able to successfully differentiate to NRPCs, AD-NRPCs ultimately fail to express CD121a, while T-NRPCs are able to expresses CD121a at significantly high levels (e.g., 90%). Thus, of the various NRPCs, T-NRPCs are able to express CD121a to effect improvement in treating damaged nerve cells.

### Selection of NRPC-Candidates Expressing CD121a Above Threshold Levels is Key to Generating Effective NRPC Products

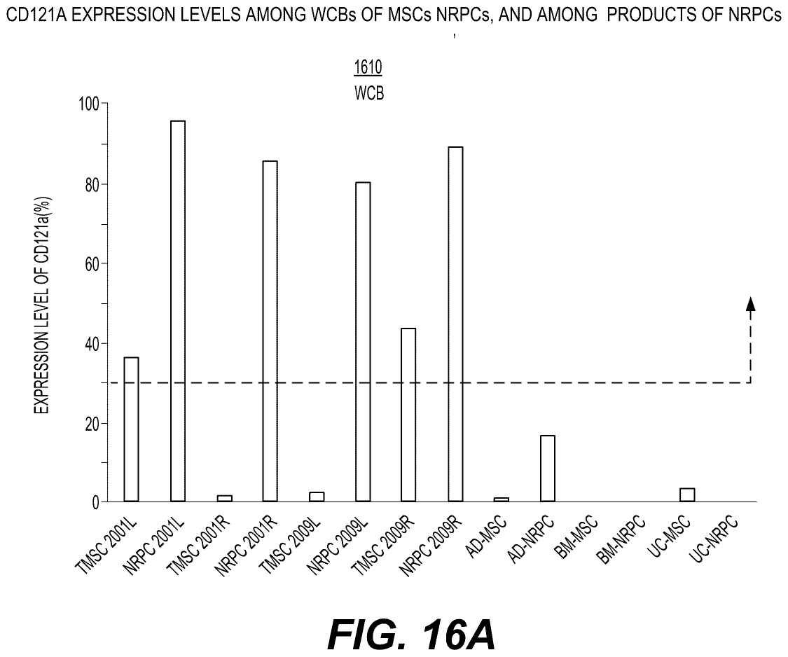

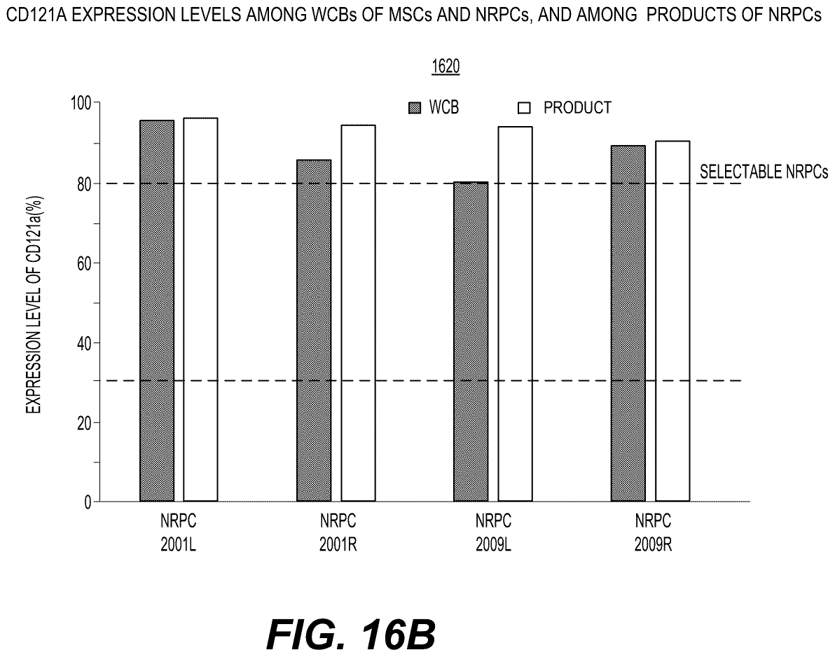

As previously discussed, in relation to FIGS. 10A and 10B, shown also in graph 1610 of FIG. 16A, expression levels of the marker CD121a vary among MSC and NRPC samples obtained from working cell banks, where the MSCs and NRPCs are derived from various regions. Among these sample MSCs and NRPCs in the working cell bank stage of the NRPC production process (e.g., at passage 9), the inventors discovered that selecting those samples (NRPC-candidates) expressing CD121a above at or above a first expression level threshold (e.g., 30%) leads to products of NRPCs that express CD121a at significantly higher levels at later stages, and therefore are significantly more effective at nerve regeneration. The samples selected—T-NRPC 2001L, T-NRPC 2001R, T-NRPC 2009L, and T-NRPC 2009R—expressed CD121a at least the first expression level threshold of 30%. Furthermore, these selected T-NRPC samples from the working cell bank stage were ultimately able to express CD121a at or above a second expression level threshold (e.g., 80%) when these samples were in their product stage. However, some samples, which expressed CD121a levels below the threshold were not selected. Thus, in some embodiments, CD121a expression levels of 30% or more among NRPC samples in the working cell bank (WCB) stage can be used as a criteria (referred to herein as “first threshold,” “first expression level,” “first expression rate,” or “first expression level threshold”) for determining which of these NRPC samples (referred to herein as NRPC-candidates) are to be selected to be used for the rest of the production process in forming the product of NRPCs. However, in some embodiments, the first threshold for CD121a expression levels may be higher. For example, NRPC-candidates may be selected if their CD121a expression levels are at least above a threshold, which is 30%, 35%, 40%, 45%, 50%, 55%, 60%, 65%, 70%, 75% or 80%. In embodiments, the threshold may be within a range formed by selecting any two numbers (two percentages) listed in the immediately previous sentence (e.g., an NRPC candidate sample may be selected if the CD121a expression rate that is at least above a threshold, where the threshold is about 30%, between about 30% and about 40%, between about 40% and about 60%, etc.). In some embodiments, the product NRPCs can be further screened for samples expressing CD121a above a second expression level threshold (e.g., 80% or more). For example, in some embodiments, the methods for generating effective NRPCs may involve selecting NRPC-candidates from the working cell bank that meets the first threshold for CD121a expression (e.g., 30% or more) and then selecting, from NRPCs in the product stage, those NRPCs that express CD121a above a second threshold (e.g., 80% or more after being thawed or placed in a live standard). Even at the product stage, the inventors discovered that as T-NRPC samples were thawed and subcultured for one or more passages, the CD121a expression rates increased even further (e.g., to 90%). The expression rates for the NRPC candidates selected from the working cell bank stage, and the expression rates for NRPCs selected from the product are thus shown side in graph 1620 of FIG. 16B.

### The Improved NRPCs were Tested in Mice that were Prepared to Simulate Critical Limb Ischemia (CLI)

As will be discussed herein, the improved NRPCs described herein were tested in mice simulating critical limb ischemia (CLI) and produced favorable results. CLI is a debilitating disease where fatty deposits form on the arterial walls of legs, causing atherosclerosis and reducing blood flow. As a result, inflammation and necrosis may occur in the tissues of the leg region. In severe cases, about 40% of patients have their limbs amputated, and about 20% of patients die within 6 months. To test the efficacy of the improved NRPCs disclosed herein, mice were prepared to simulate patients having CLI. Specifically, the femoral artery on the leg side of the mouse was ligated and removed to prepare an animal model of the same morbid state as patients having CLI.

### The Experiment for Testing the Effect of the Improved NRPCs Involved Several Administration and Control Groups

For the experiment, seven groups of mice were prepared to simulate CLI, of which six received varied treatments (the six groups referred to herein as administration groups). One of the seven groups prepared to simulate CLI did not receive any treatments, and is referred to herein as the negative control group. Furthermore, one group of mice was normal (i.e., not prepared to simulate the CLI) and is referred to as the normal group. Clopidogrel (CP), a drug for the treatment of CLI, was selected for administration in one of the six administration groups for the experiment. At a dose of 0.025 mg/20 g, CP was administered in 5 parts into the leg muscles of mice whose blood vessels had been removed. Four of the six administration groups of mice were administered MSCs and NRPCs. Specifically, one administration group received T-MSCs, another received AD-MSC, another received T-NRPC, and another received AD-NRPC. The administered NRPCs in two of the administration groups were differentiated from their respective MSCs, which were being administered in two administration groups. For the remaining administration group, the mice received an excipient (CS10). For the administration groups receiving the MSCs and NRPCs, a large number (e.g., 1×10⁶) of the respective cells were divided and administered to 5 leg muscle regions of the mice in which blood vessels were removed. For each of the experimental groups, blood flow into the leg of the mouse (measured by a percentage of blood perfusion) was analyzed for 3 weeks on a weekly basis. For the groups where the mice was prepared to simulate CLI, the leg corresponded to the one in which blood vessels were removed.

### The Results of the Experiment on the Animal Model of CLI Showed that T-NRPC Improved Blood Flow More Effectively Compared to Other Treatments

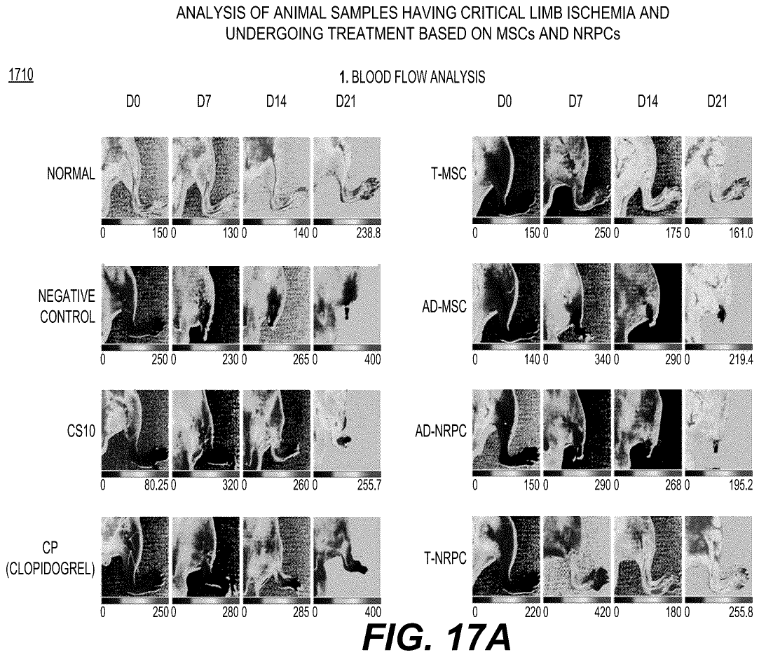

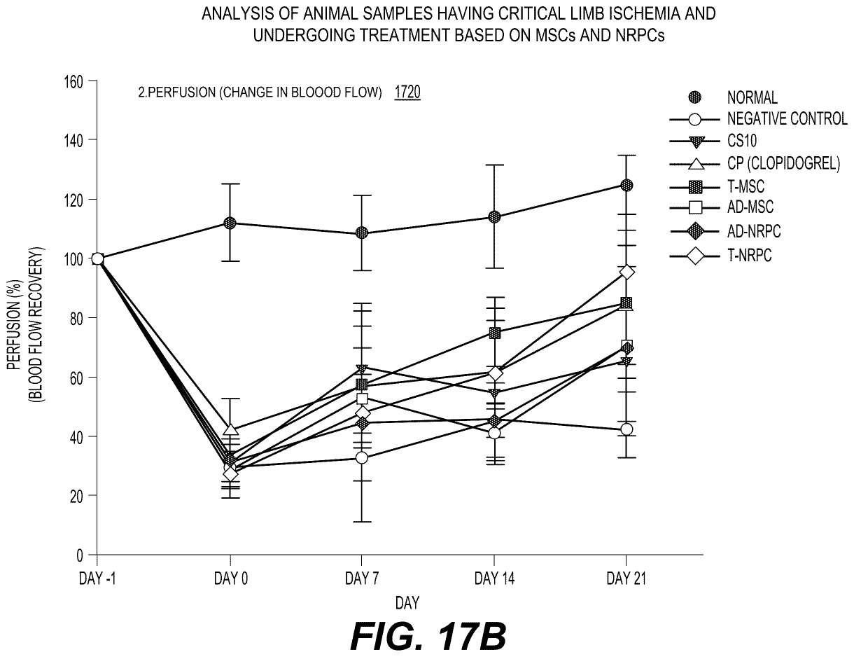

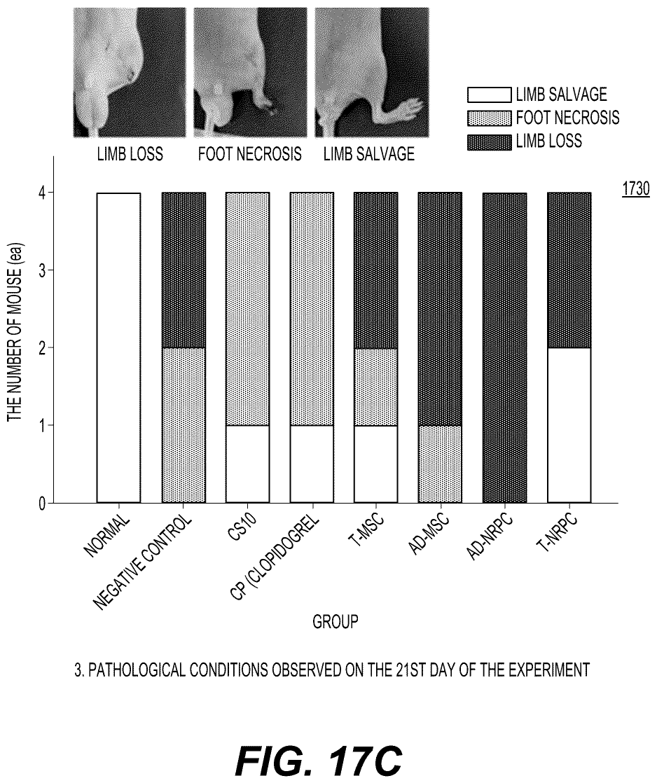

Over the three weeks (e.g., the 21 days), blood perfusion was measured across the eight groups of the above described experiment, and the pathological symptoms of leg necrosis and amputation due to inflammation were determined through visual observation. FIGS. 17A to 17C show the results of the experiment. Specifically, the set of images 1710 of FIG. 17A illustrates blood flow analysis over a period of time in across all eight groups of the animal samples, including the seven groups prepared to simulate CLI and the six administration groups undergoing various forms of treatment based on MSCs, NRPCs, CS10, and CP. As shown in these set of images 1710, which shows blood perfusion over the 21 days (D0 through D21), blood flow (show in red) increased in the group administered with T-NRPC compared to groups administered with the inducer excipient (CS10), Clopidogrel (CP), T-MSC, AD-MSC, and AD-NRPC. Thus, the inventors discovered that the pathological symptoms caused by lower extremity necrosis and the risk of amputation decreased in the group administered with T-NRPC. FIG. 17B includes a graph 1720 quantifying the change in blood flow across the 21 days for all eight groups using a program dedicated to Laser Doppler Image. FIG. 17C includes a graph 1730, which indicates the pathological conditions (e.g., necrosis, amputation, and survival) observed on the mouse leg for all eight groups on the 21st day of the experiment. The white bar indicates the number of intact mouse legs (e.g., no necrosis or amputation caused by the administered substance); the dark gray bar indicates the number of animals whose mouse legs were amputated; and the light gray bar indicates the number of animals with necrotic mouse limbs. As shown in graph 1730, the group administered with T-NRPC had a higher percentage of intact mouse legs compared to all other groups except for the normal group. Thus, the animal studies revealed that the improved T-NRPC of the present disclosure is effective at treating diseases such as CLI.

### T-NRPCs were Also Effective at Curtailing Muscle Fibrosis, Reducing Muscle Inflammation, and Forming Capillaries in the Animal Models

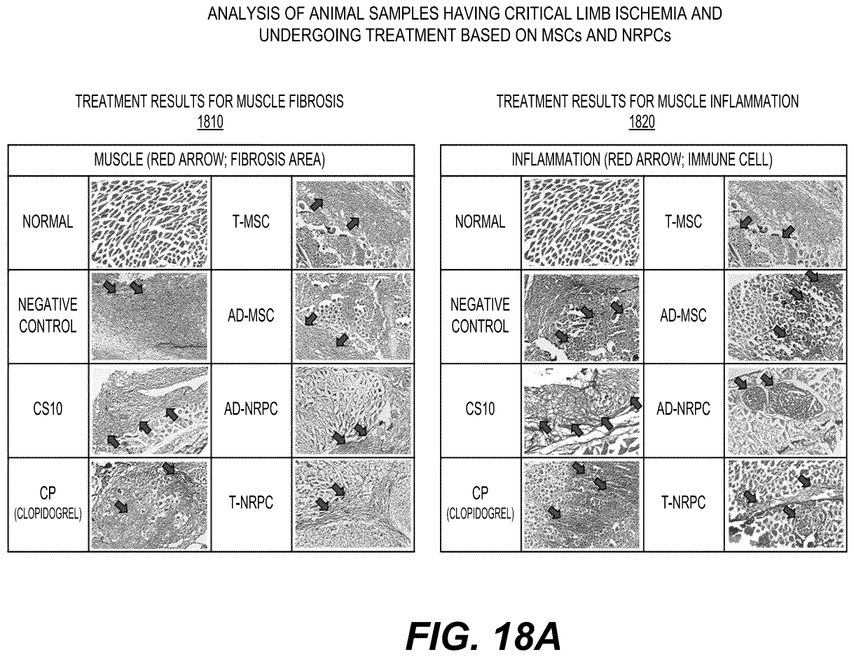

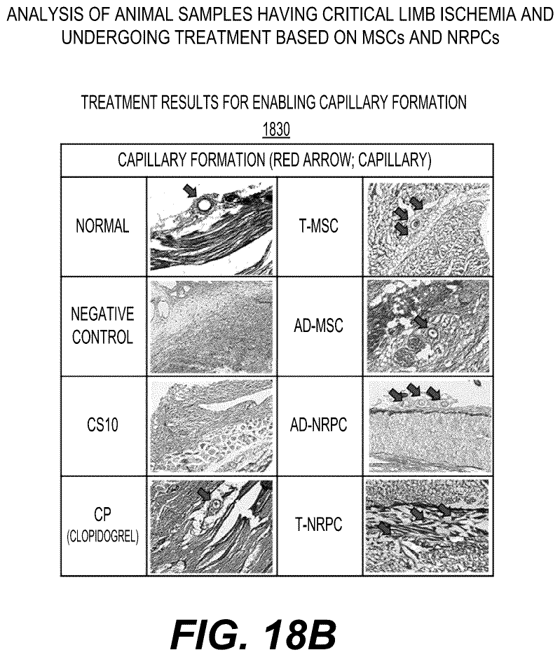

The various treatments administered to the animal models of CLI were also investigated for their ability to treat muscle fibrosis, muscle inflammation, and capillary formation over the course of the 21 days. As previously discussed, the eight experiment groups of mice from the foregoing experiment were the normal group (i.e., mice not prepared to simulate the CLI), the negative control group (i.e., mice prepared to simulate CLI and not receiving any treatment), and the six administration groups of mice prepared to simulate CLI, the six administration groups including groups receiving CS10, CP, T-MSC, AD-MSC, AD-NRPC, and T-NRPC. FIGS. 18A and 18B show sets of images illustrating varying degrees of muscle fibrosis, muscle inflammation, and capillary formation on after the treatment period (e.g., at the 21^{st} day after treatment was rendered in the administration groups). Specifically, set 1810 shows the results of treatment towards muscle fibrosis, set 1820 shows the results of treatment towards muscle inflammation, and set 1830 shows the results of treatment towards capillary formation. As shown in FIGS. 18A and 18B, in the group where T-NRPCs were administered, muscle fibrosis progressed less and showed histological findings in which the shape of the muscle was maintained. The T-NRPC administered group also showed the lowest inflammatory areas and high angiogenesis. Thus, the inventors discovered that T-NRPCs have therapeutic effects in various areas, such as anti-inflammatory effect, inhibition of myofibrosis, and neovascularization. Therefore, the T-NRPCs disclosed herein have high therapeutic applicability for critical limb ischemic disease.

### Experimental Results Showed that T-NRPCs Prevent Muscle Fibrosis Progression

In order to assess the state of muscle fibrosis across the eight experiment groups, tissue from the leg of the mouse was removed and histological pathology was analyzed on the 21^{st} day of the experiment (e.g., after the three week treatment time of the experiment). As shown in set 1810, the image of the Normal group most closely resembles the image of the group administered with T-NRPC, as the muscle fiber bundles in both images are distributed in an oval or circular shape. However, the shape of the muscle fiber bundles in the negative control group shows a general form of muscle necrosis in which muscle fiber bundles are not visible and the shape of the muscle fiber is crushed. The groups treated with CS10, CP, T-MSC, and AD-MSC showed a lower level of muscle fiber recovery.

### Experimental Results Showed that T-NRPCs Reduce Muscle Inflammation

In order to assess muscle inflammation across the eight experiment groups, tissue was removed from the leg of the mouse and the degree of inflammatory cell infiltration was analyzed through histological pathology on the 21^{st} day of the experiment (e.g., after the three week treatment time of the experiment). As shown in set 1820, areas suffering from inflammation are stained a darker shade of purple and are infiltrated with inflammatory cells distributed between muscle fibers or between muscle masses. In the case of the normal group, no signs of inflammation were seen. The negative control group had the largest distribution of inflammatory cells. For groups treated with CS10, CP, T-MSC, AD-MSC, and AD-NRPC, there was less inflammation compared to the negative control group, but more compared to the group administered with T-NRPC.

### Experimental Results Showed that T-NRPCs Facilitate Capillary Formation

In order to assess capillary formation across the eight experiment groups, tissue from the leg of the mouse was removed and the degree of blood vessel formation was analyzed through histological pathology on the 21st day after administration of the experiment (e.g., after the three week treatment time of the experiment). As showed in set 1830, since blood vessels contain endothelial cells and red blood cells, the histology results show that red blood cells are gathered around in a circular shape. In the normal group, the largest blood vessel in the leg of the mouse was that of the femoral artery. In the negative control group and the group treated with CS10, no blood vessels were observed. However, angiogenesis was confirmed in the CP, T-MSC, AD-MSC, AD-NRPC, and T-NRPC administration groups.

### The T-NRPCs were Also Investigated for their Use in Treating Charcot-Marie-Tooth Disease Using a Nerve Conduction Test

Charcot-Marie-Tooth (CMT) disease is a hereditary disease that occurs in 1 in 2,500 people, and the phenotype and genetic causes are heterogeneous. CMT type 1A (CMT1A) is a type of inherited neurological disorder that affects the peripheral nerves and is caused by a duplication of the gene for peripheral myelin protein 22 (PMP22). The inventors investigated whether the MSCs and NRPCs disclosed herein (e.g., the improved T-NRPCs disclosed herein) were effective at treating CMT1A. In an experiment, the C22 mouse was used. The C22 mouse is a mouse that has 7-8 copies of human PMP22, including about 40 kb in the proximal region, and is often used for Charcot-Marie-Tooth disease research. Five randomly selected groups of 5-week-old C22 mice were used for the experiment. Of the five groups, three groups received performed subcutaneous injection with varying doses of T-NRPC. Specifically, one group received a low dose of T-NRPC (referred to herein as NRPC-L), comprising about 2.5×10\hat{ }4 NRPCs; another group received a medium dose of T-NRPCs (referred to herein as NRPC-M), comprising about 2.5×10\hat{ }5 NRPCs; and another group received a high dose of T-NRPCs (referred to herein as NRPC-H), comprising about 5×10\hat{ }5 NRPCs. The T-NRPCs were administered to the group twice, where the second dose of T-NRPCs was administered 4 weeks after the first administration. Another group received a percutaneous injection of CS10 (the group referred to as “sham”), while the remaining group was not administered anything (the group referred to as wild type (W/T)).

### T-NRPCs Administered at Higher Doses Improved Nerve Conduction in Mice

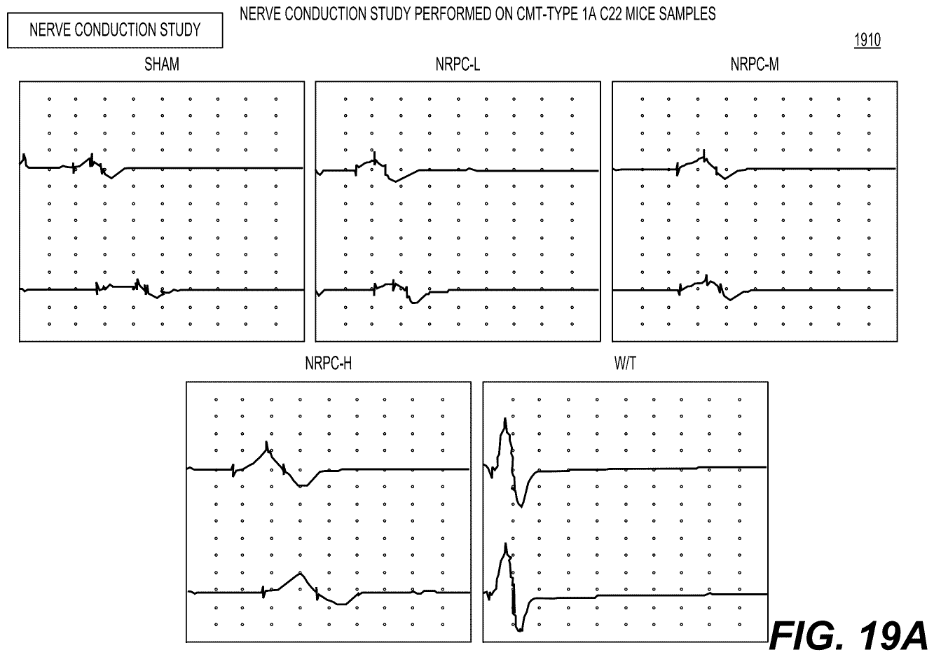

At 16 weeks, a nerve conduction test was performed to obtain a proximal response to the calf muscle (biceps femoris), and histological analysis was performed by tissue excision. FIGS. 19A to 19C illustrate the results of the nerve conduction study performed on mice samples, through graphs 1910, 1920, and 1930. Specifically, graph 1910 shows the amplitude of the waveform associated with the nerve conduction from the calf muscle (biceps femoris). Graph 1920 shows the nerve conduction velocity during the nerve conduction from the calf muscle (biceps femoris). As shown in graph 1920, the nerve conduction velocity significantly increased in C22 mice transplanted with T-NRPCs at higher doses (e.g., NRPC-H) compared to C22 mice transplanted with T-NRPCs at lower doses or the Sham group. Graph 1930 shows the compound muscle action potential (measured in mV) associated with the nerve conduction. Here again, improvement in CMAP is seen with C22 mice transplanted with the T-NRPC in higher doses (e.g., NRPC-H) compared to C22 mice transplanted with T-NRPCs at lower doses or the Sham group.

### The T-NRPCs Also Increased Myelination of Nerves of the C22 Mice

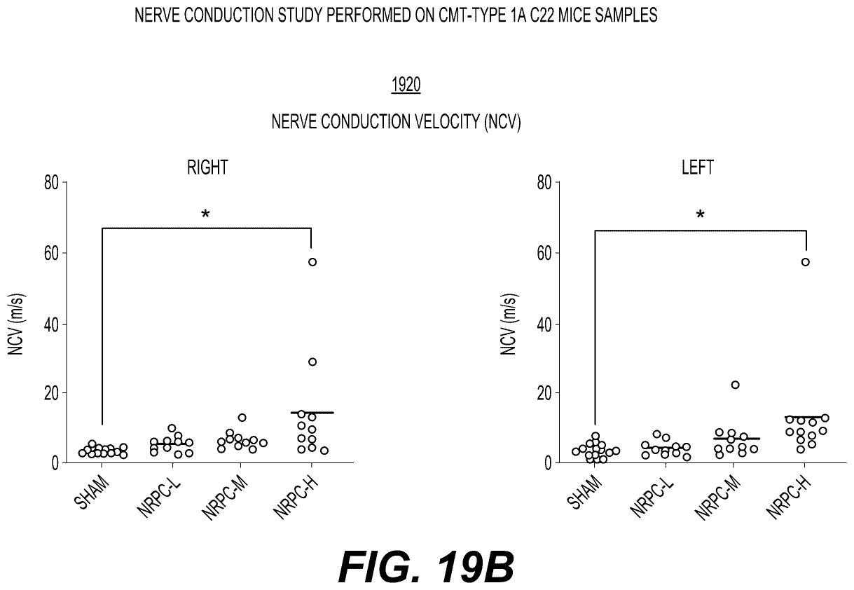

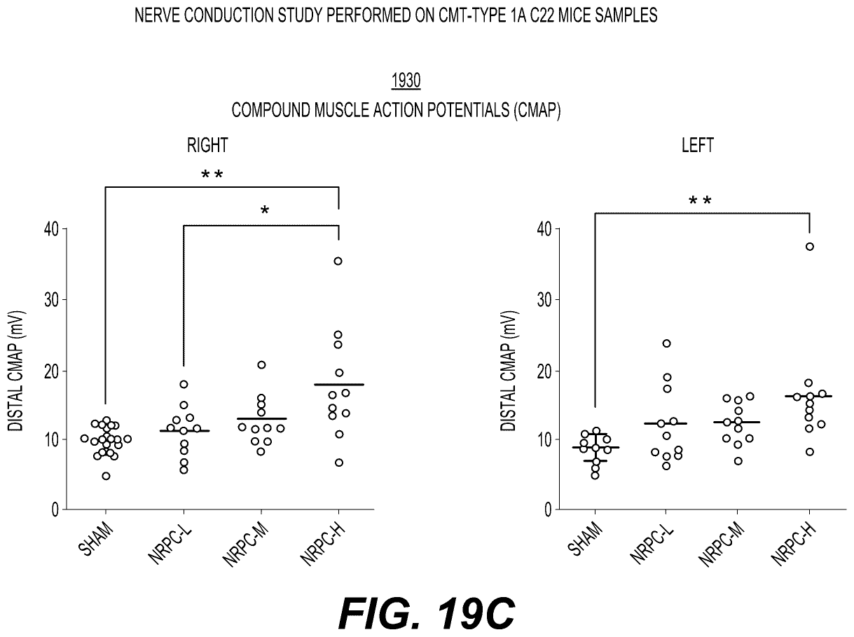

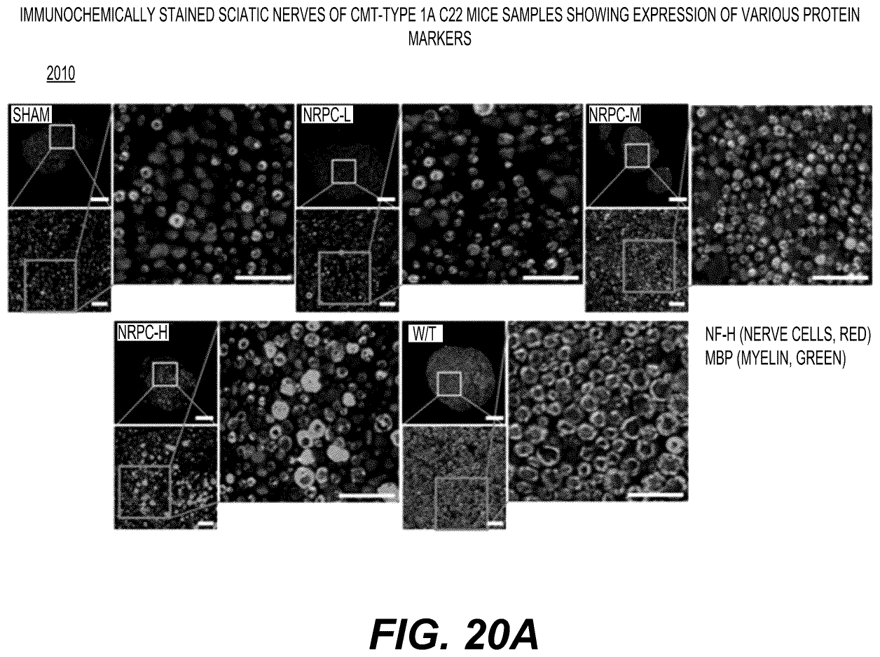

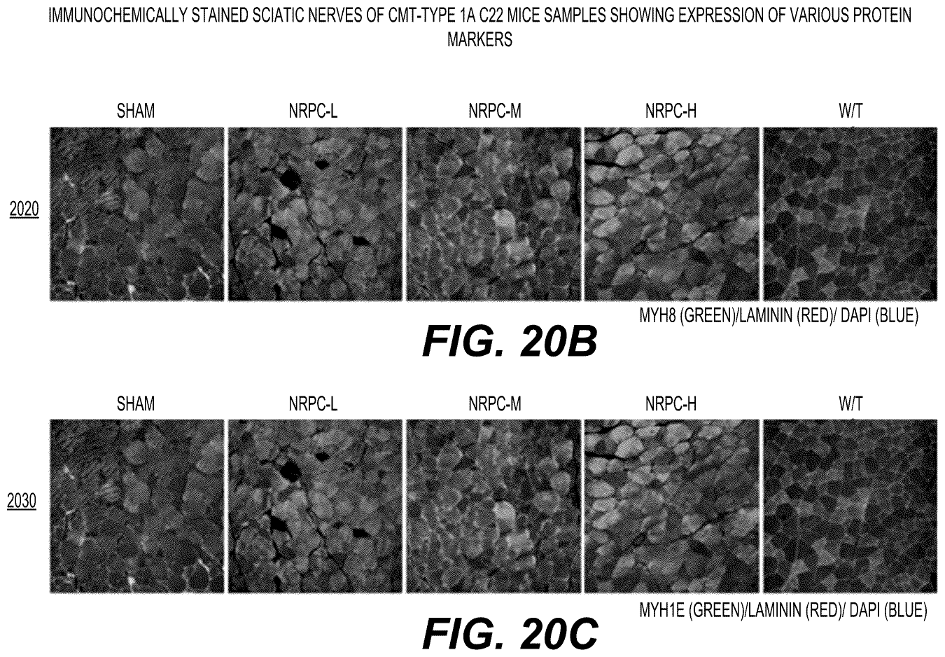

The varying levels of T-NRPCs administered to the groups of C22 mice were also found to increase myelination of the nerves. FIGS. 20A to 20C show three sets of images of immunochemical stained sciatic nerves of the five C22 mice groups from the above experiment, where three of the C22 mice groups were treated using varying levels of T-NRPCs, (NRPC-H, NRPC-M, and NRPC-L), as described above. The varying levels of T-NRPC resulted in varying levels of protein markers useful for myelination, as predicted by the experiments performed by the inventors and previously described herein. The first set of images 2010 shows that the C22 mice groups transplanted with varying levels of T-NRPCs showed increased expression of proteins that form myelin (e.g., MBP, shown in green) surrounding proteins that are precursor for nerve cells (e.g., NF-H, shown in red). Moreover, the expression of MBP (green) and NF-H (red) increased as the amount of T-NRPC administered to the C22 mice increased, indicating that the formation of axons and myelin of the sciatic nerve of C22 mice can be improved with T-NRPCs.

### The T-NRPCs Also Improved Muscle Regeneration in the C22 Mice

The experiment also tested, by detecting from the immunochemical stained images, the expression of MYH8, a marker protein for regenerative muscle, and laminin, a basement membrane protein of the extracellular matrix. As shown in the second set of images 2020 of FIG. 20B, there was an increased expression of these proteins in the C22 mice groups injected with T-NRPCs. The shape of muscle fibers in the groups treated with T-NRPCs were maintained and restored at a level similar to W/T group. The third set of images 2030 show the expression of MYH1E and laminin, which are both skeletal muscle proteins. Here again, the C22 mice groups treated with NRPCs tended to have more expressions of MYH1E and laminin. Moreover, the shape of the muscle fiber in the NRPC-H group was improved to a degree similar to that of the W/T group.

### T-NRPCs were Also Found to Remyelinate Damaged Nerves

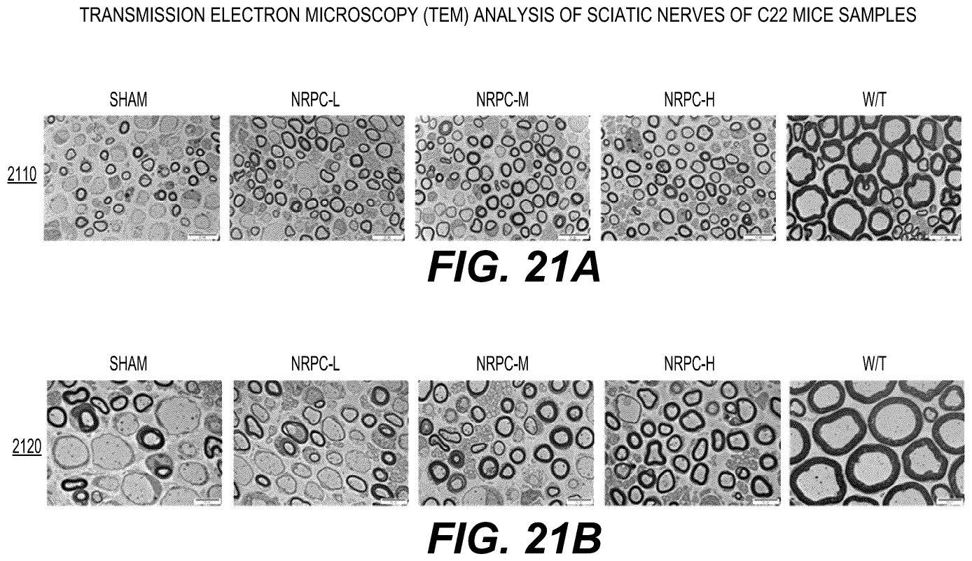

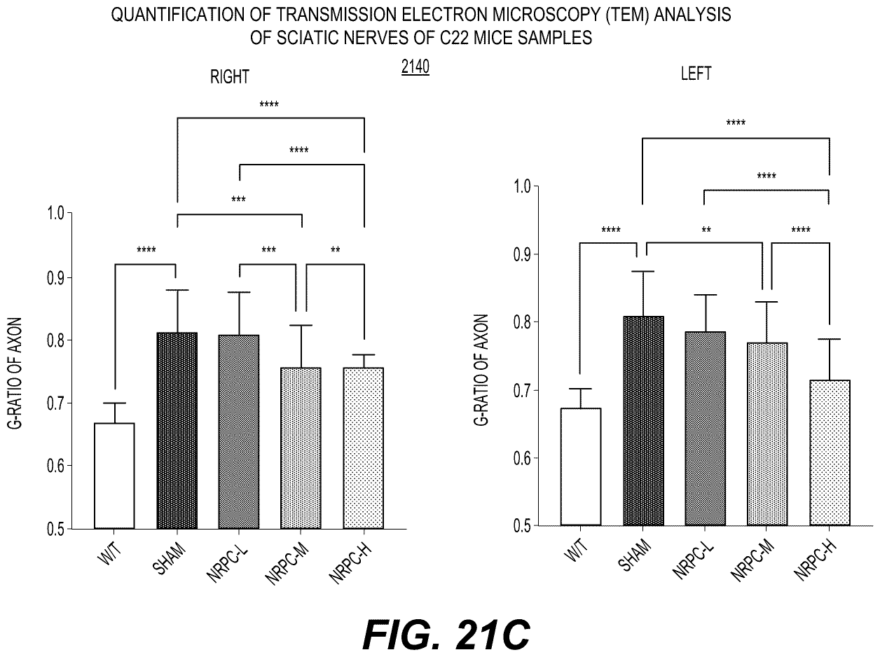

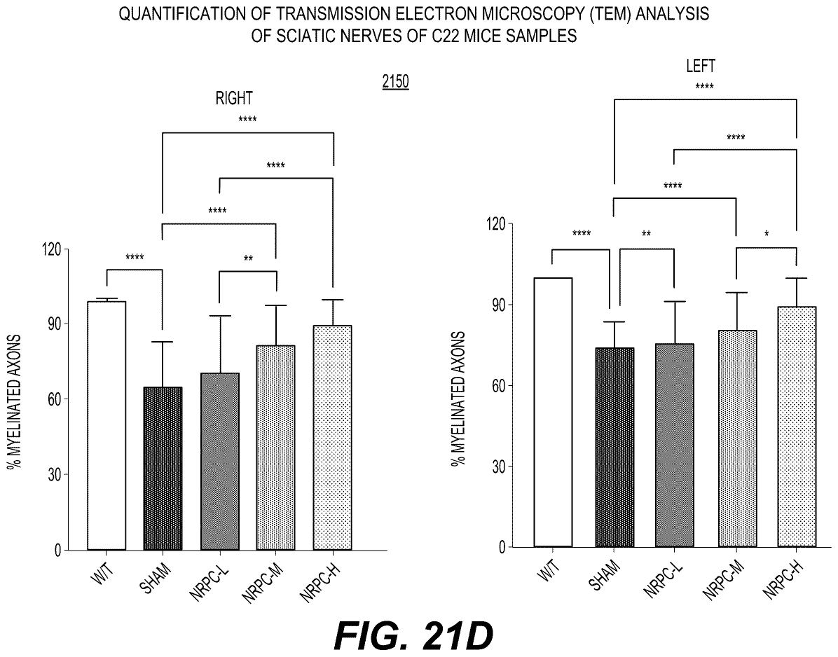

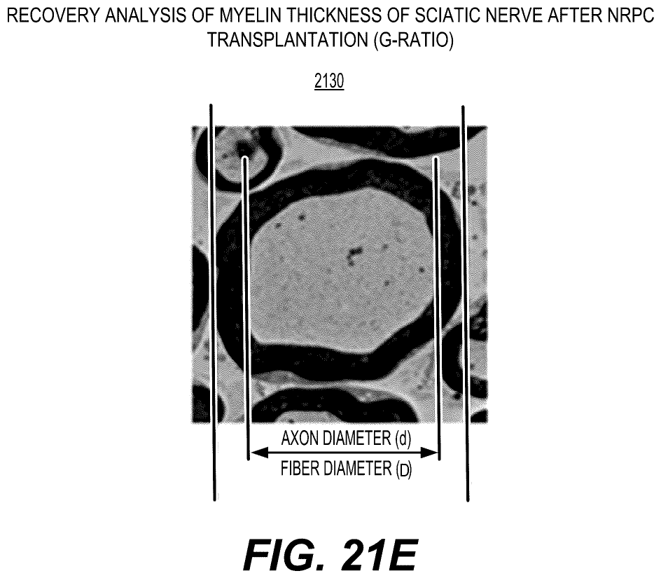

The varying levels of T-NRPCs administered to the groups of C22 mice were also found to remyelinate nerves (e.g., where myelin was destroyed). FIGS. 21A to 21E show five sets of images indicating the G-ratio and myelination of neurons of the five C22 mice sample groups treated, including the three groups treated using varying levels of T-NRPCs (NRPC-H, NRPC-M, and NRPC-L). In the first set of images 2110, transverse sections of the mouse sciatic nerve were observed with an electron microscope (TEM) to morphologically observe the remyelination of axons according to the high, medium, and low concentrations of T-NRPCs that were administered. (×3,000). In the transverse section of the nerve fiber of the sciatic nerve, a round axon and the myelin sheath surrounding the axon can be observed. The second set of images 2120 shows the results after the treatments were rendered. As shown through both sets of the images, the samples administered with higher concentrations of T-NRPC exhibited remyelination of axons, resembling closest to the W/T (normal) group. For example, in the W/T group, the myelin surrounding the axon can be observed normally. On the other hand, in the Sham group, there is almost no myelin surrounding the axon. In the group treated with NRPCs at low, medium, or high concentrations, the formation of myelin surrounding the axon can be observed. The third set 2130 is a single image explaining the G-ratio measurement method in the nerve fiber image of the electron microscope. The fourth set of images 2140 shows that the Axon G-ratio was improved with increasing levels of T-NRPC in the C22 mice, as shown by the increasing G-ratios exhibited by the NRPC-L, NRPC-M, and NRPC-H groups, respectively, with the NRPC-H group exhibiting a G-ratio closest in value to the W/T (normal) group. The fifth set of images 2150 show that, in the C22 mice groups where NRPC was administered at low, medium, and high concentrations, respectively, the myelin thickness increased with increased T-NRPC. Thus, the experiments show that T-NRPCs increase myelination of nerve cells and remyelination of the destroyed myelin.

### T-NRPCs were Also Found to Express MPZ while Regulating PMP22 to Optimal Levels

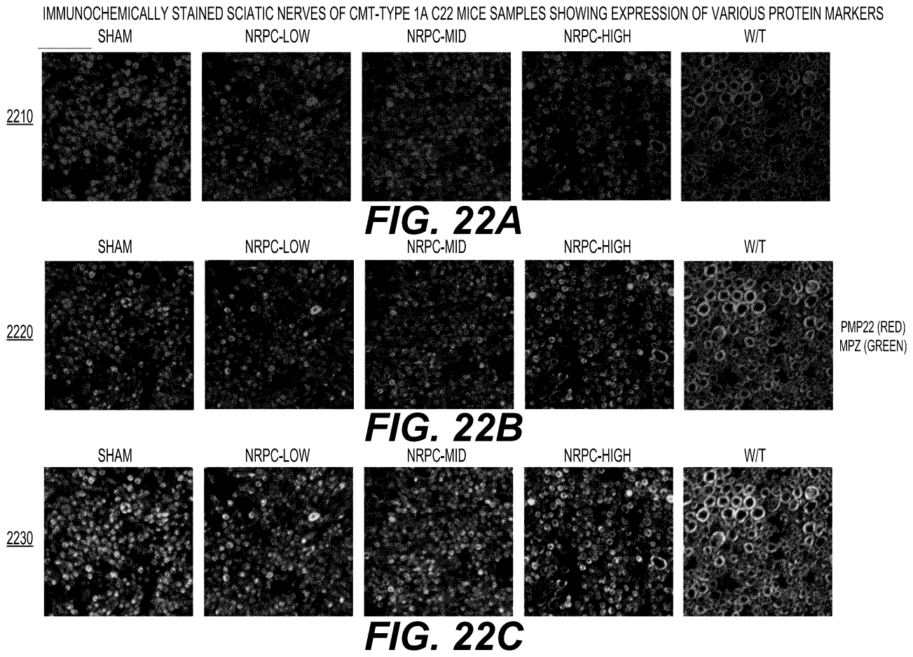

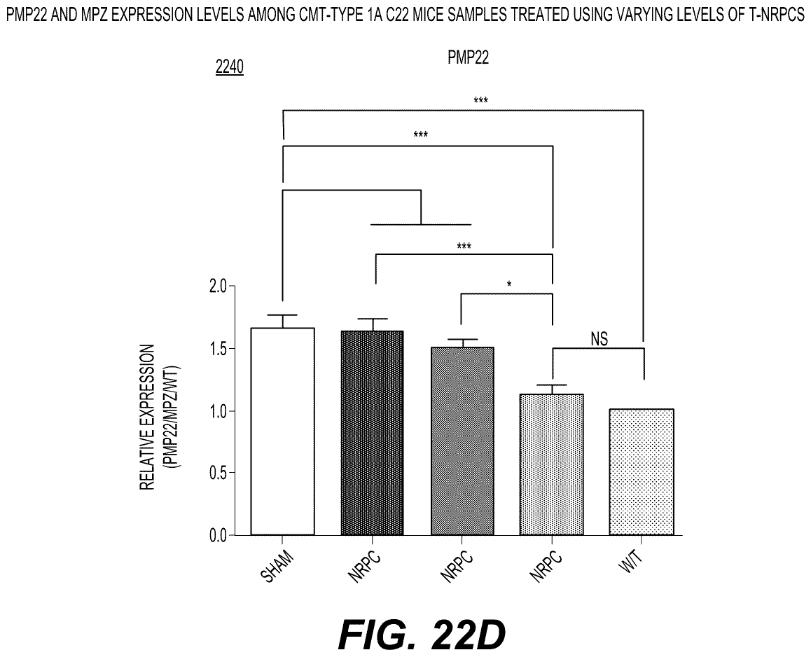

As previously discussed, CMT type 1A (CMT1A) is caused by a duplication of the gene for peripheral myelin protein 22 (PMP22). Although the expression of PMP22 is important for myelination in the developmental process of the sciatic nerve, an overexpression of PMP22 can induce CMT1A and demyelinating neuropathy. Therefore, PMP22 needs to be downregulated if overexpressed. Meanwhile, myelin protein zero (MPZ) is another protein important for myelin development. Specifically, MPZ is important for the formation and stabilization of multilamellar structures of myelin and is a key marker for Schwann cell development. During healthy conditions, it is ideal to have PMP22 expression proportionate to MPZ expression. The inventors investigated whether the T-NRPCs of the present disclosure had the potential to express and regulate levels of PMP22 and MPZ in the C22 mice groups to which the T-NRPCs were administered in varying amounts (NRPC-H, NRPC-M, and NRPC-L). FIGS. 22A to 22C shows three set of images and a graph indicating the expression of markers PMP22 and MPZ among the C22 mice samples. While the first set of images 2210 shows the expression levels of PMP22 in red; the second set of images 2220 shows the expression levels of MPZ in green; the third set of images 2230 is a composite of sets 2210 and 2220, thereby showing whether the expression of MPZ and PMP22 are balanced; and the graph 2240 (FIG. 22D) indicates the expression ratio of PMP22/MPZ for each of the C22 mice groups.

The PMP22/MPZ ratio was 1 in the W/T group. On the set of images 2230 that formed a composite of sets 2210 and 2230, the Sham group's orange color, which indicated a higher PMP22 presence, contrasted with the yellow color of the W/T group and the groups administered with the T-NRPCs, which indicated a more balanced portion of PMP22 and MPZ. Thus, the results showed that T-NRPCs can effectively promote the recovery of Schwann cells and the downregulation of PMP22 by improving the PMP22/MPZ ratio to a value similar to that of the W/T (normal) group.

### The T-NRPCs were Able to Regulate PMP Expression by Expressing miR-29a

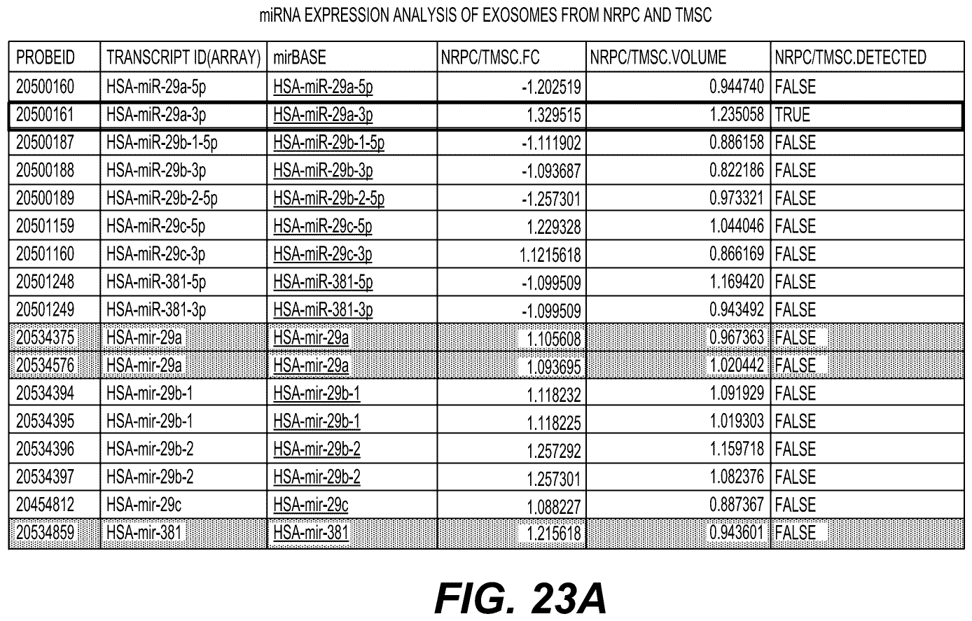

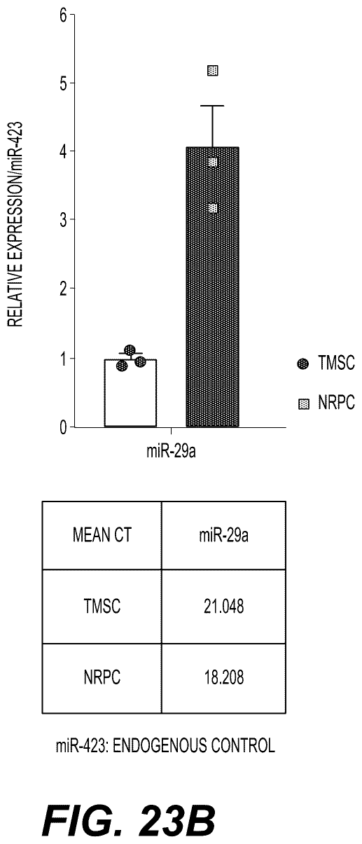

The inventors also investigated why the NRPC treated C22 mice groups were able to regulate the overexpression of the PMP22 gene. In order to confirm the mechanism for the regulation of PMP22 expression by T-NRPCs, various micro RNAs (miRNAs) of exosomes obtained from TMSC and NRPC cultures were analyzed. FIG. 23A is a table illustrating the analysis of several miRNAs to determine which appeared to be expressed in TMSC and T-NRPC cultures. The inventors discovered that expression of the miRNA identified as miR-29a was higher in the T-NRPC than in T-MSC cultures. miR-29a is a microRNA that regulates PMP22 expression. Therefore, the inventors discovered that T-NRPCs are effective at treating CMT disease as T-NRPCs can be used to suppress PMP22 overexpression via expression of miR-29a. Thus, when such T-NRPCs were administered to the C22 mouse model, which is an animal model having the highest incidence rate for CMT disease, the disease being characterized by overexpression of the PMP22 gene, the inventors confirmed that PMP22 gene expression was regulated and CMT disease was improved. The inventors found that the T-NRPCs were characterized by an increased expression of miR-29a, and this increased expression of miR-29a allowed the T-NRPCs to inhibit PMP22 gene expression in the C22 mouse, thus treating the CMT disease.

Expression of miR-29a in the T-NRPCs

As shown in FIGS. 23A and 23B, the inventors found that the T-NRPCs (e.g., for use as a product) were characterized by an increased expression of miR-29a, as evident from having 1.329515 in fold change (fc) value and 1.235058 in volume. The fold change value (fc) indicates a ratio of expression of a given microRNA (e.g., miR-29a) in T-NRPC over the expression of the microRNA in the corresponding T-MSC. Thus, as indicated in FIGS. 23A and 23B, miR-29a is expressed 1.329515 times more in T-NRPC than in T-MSC. In addition, the volume indicates a ratio of expression intensity of a given microRNA (e.g., miR-29a) in T-NRPC over the expression intensity of the given microRNA (e.g., miR-29a) in the corresponding T-MSC. Thus, as indicated in FIGS. 23A and 23B, the expression intensity of miR-29a is 1.235058 times higher in T-NRPC than in T-MSC. In some embodiments, T-NRPCs may be selected (e.g., for making the product) based on a fold change value (fc) of those T-NRPCs expressing miR-29a (when compared to the expression of miR-29a in the respective T-MSCs) as at least above a threshold that is about 1.1, 1.15, 1.2, 1.25, 1.3, 1.35, 1.4, 1.45, 1.5, 1.55, or 1.6. In some embodiments, the threshold may be within a range formed by selecting any two numbers (two fold change values (fcs)) listed in the immediately previous sentence (e.g., the T-NRPCs may be selected based on a fold change value (fc) of those expressing miR-29a being above a threshold that is between about 1.1 and about 1.5, between about 1.15 and about 1.25, etc.). In some embodiments, T-NRPCs may be selected (e.g., for making the product) based on a miR-29a volume of the T-NRPCs (i.e., ratio of expression intensity of miR-29a in the T-NRPCs over the expression intensity of miR-29a in the respective T-MSCs) being at least above a threshold that is about 1.0, 1.05, 1.1, 1.15, 1.2, 1.25, 1.3, 1.35, 1.4, 1.45, or 1.5. In some embodiments, the threshold may be within a range formed by selecting any two numbers (two volumes) listed in the immediately previous sentence (e.g., the T-NRPCs may be selected based on a volume being above a threshold that is between about 1.0 and about 1.5, between about 1.15 and about 1.25, etc.). FIGS. 23A and 23B further show the use of miR-413 as an endogenous control that may have a constant expression rate, and may be used for quantitative comparison of the amount of miR-29a expressed in T-MSC and T-NRPC. For example, as shown in FIGS. 23A and 23B, the expression rate of miR-29a (as measured by the fold change value (fc) and the volume) compared to the expression rate of miR-413 was about 4 times higher in T-NRPC than in T-MSC.

The inventors also found the average (mean) threshold of cycle (Ct) values for miR-29a. It is contemplated that if there are more target genes, the mean Ct value would be faster, but if there are fewer target genes, the mean Ct value would be slower. The inventors found the mean Ct value for miR-29a in T-NRPC to be 18.208, which was faster than the mean Ct value for miR-29a in T-MSC, thus finding that more miR-29a was present in T-NRPC.

### The T-NRPCs are Effective for the Treatment of any Ischemic Tissue

Although the above described animal studies were performed on Balb/c nude mice simulating CLI, it is contemplated that the disclosed T-NRPCs will be able to provide the disclosed advantageous effects on any ischemic tissue. As generally known, ischemia refers to a restriction in blood supply to any tissue, muscle group, or organ of the body, causing a shortage of oxygen to the tissue. As a result of ischemia, the damaged tissue may result in damaged nerves, (e.g., demyelination and improper nerve conduction), muscle fibrosis, muscle inflammation, and reduced blood flow, characteristic of the nerves and tissues described in FIGS. 19-23. The advantageous effects that T-NRPCs deliver to the ischemic tissues of the lower limb of the C22 mice can thus be applied in other ischemic tissues and/or to other animals (e.g., humans).

### Administration and Formulation of the Compositions Described Herein

The pharmaceutical composition of the present disclosure may be administered orally or parenterally. Specifically, it may be administered parenterally, e.g., by intravenous injection, transdermal administration, subcutaneous injection, intramuscular injection, intravitreal injection, subretinal injection, suprachoroidal injection, eye drop administration, intracerebroventricular injection, intrathecal injection, intraamniotic injection, intraarterial injection, intraarticular injection, intracardiac injection, intracavernous injection, intracerebral injection, intracisternal injection, intracoronary injection, intracranial injection, intradural injection, epidural injection, intrahippocampal injection, intranasal injection, intraosseous injection, intraperitoneal injection, intrapleural injection, intraspinal injection, intrathoracic injection, intrathymic injection, intrauterine injection, intravaginal injection, intraventricular injection, intravesical injection, subconjunctival injection, intratumoral injection, topical injection, etc.

The administration dosage of the pharmaceutical composition of the present disclosure may vary depending on various factors such as formulation method, administration method, administration time, administration route, the response to be achieved with the administration of the pharmaceutical composition and the extent thereof, the age, body weight, general health condition, pathological condition or severity, sex, diet and excretion rate of a subject to which the pharmaceutical composition is administered and other drugs or ingredients used together and similar factors well known in the medical field, and an administration dosage effective for the desired treatment may be easily determined and prescribed by those having ordinary knowledge in the art.

The administration route and administration method of the pharmaceutical composition of the present disclosure may be independent from each other and are not specially limited as long as the pharmaceutical composition can reach the target site.

Formulations of the Compositions Described Herein for Administration into a Subject

In at least one embodiment, formulations for the finished product to be administered may be in a frozen state. In some aspects, the formulations may be thawed prior to administration, in accordance with the thawing process for cells described above. In other aspects, the formulations may be administered while in their frozen state. Formulations for the parenteral administration may include a sterilized aqueous solution, a nonaqueous solution, a suspension, an emulsion, a freeze-dried formulation and a suppository. For the nonaqueous solution or suspension, propylene glycol, polyethylene glycol, a vegetable oil such as olive oil, an injectable ester such as ethyl oleate, etc. may be used. As a base of the suppository, witepsol, macrogol, Tween 61, cocoa butter, laurin butter, glycerogelatin, etc. may be used. The pharmaceutical composition of the present disclosure may be prepared into a single-dose or multi-dose formulation. In some aspects, a pharmaceutically acceptable carrier and/or excipient may be used in the formulation according to a method that can be easily carried out by those having ordinary knowledge in the art to which the present disclosure belongs. The pharmaceutical composition according to the present disclosure may be prepared into various formulations according to common methods. The composition of the present disclosure may contain one or more known active ingredient having an effect of preventing or treating a neurological disease together with the stem cell-derived, neuronal regeneration-promoting cells having neuronal regeneration activity.

### Administration Dosage

The administration dosage of the pharmaceutical composition of the present disclosure may vary depending on various factors such as formulation method, administration method, administration time, administration route, the response to be achieved with the administration of the pharmaceutical composition and the extent thereof, the age, body weight, general health condition, pathological condition or severity, sex, diet and excretion rate of a subject to which the pharmaceutical composition is administered and other drugs or ingredients used together and similar factors well known in the medical field, and an administration dosage effective for the desired treatment may be easily determined and prescribed by those having ordinary knowledge in the art. In at least one embodiment, the administration dosage may comprise the T-NRPCs described herein in an amount that is about 1×10⁵ cells/kg to about 1×10⁷ cells/kg. In another embodiment, the administration dosage may comprise the T-NRPCs described herein in an amount of about 1×10⁴ cells/kg, 5×10⁴ cells/kg, 1×10⁵ cells/kg, 5×10⁵ cells/kg, 1×10⁶ cells/kg, 5×10⁶ cells/kg, 1×10⁷ cells/kg, 5×10⁷ cells/kg, or 1×10⁸ cells/kg. In some embodiments, the administration dosage may comprise the T-NRPCs described herein within a range formed by selecting any two numbers listed in the immediately previous sentence (e.g., the administered dosage may comprise the T-NRPCs described herein in an amount that is between about 5×10⁴ cells/kg and about 5×10⁷ cells/kg, between about 1×10⁵ cells/kg and about 1×10⁶ cells/kg, etc.). In some aspects, the amount of the T-NRPCs described herein in the administration dosage may be dependent upon a variety of factors, including the age, weight, and sex of the subject, the disease to be treated, and the extent and severity thereof. In some embodiments, the administration dosage may contain the T-NRPCs described herein as an active ingredient that is about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18% 20%, 22%, 24%, 26%, 28% 30%, 32%, 34%, 36%, 38% 40%, 42%, 44%, 46%, 48% 50%, 52%, 54%, 56%, 58% 60%, 62%, 64%, 66%, 68% 70%, 72%, 74%, 76%, 78% 80%, 82%, 84%, 86%, 88%, 90% or 92% of the volume of administration dosage. In some embodiments, the threshold percentage volume for the may be within a range formed by selecting any two numbers (two percentage volumes) listed in the immediately previous sentence (e.g., the administered dosage may have the T-NRPCs described as an active ingredient that is at least a threshold that is between about 0.2% and about 2%, between about 1% and about 15%, between about 2% and about 10%, etc.)

### EXAMPLES

### Example 1.1—Obtaining T-MSCs

Left and right tonsil tissues derived from many donors acquired from Ewha Womans University College of Medicine were separated and put in a tube holding 10 mL of DPBS (Dulbecco's phosphate-buffered saline) supplemented with 20 μg/mL gentamicin, centrifuged at 1,500 rpm for 5 minutes and then washed twice. The washed tonsil issues were sliced using sterilized scissors.

In order to isolate tonsil-derived mesenchymal stem cells from the tonsil issues, after adding an enzymatic reaction solution of the same weight, the tonsil issues were incubated in a shaking incubator at 37° C. and 200 rpm for 60 minutes. The composition of the enzymatic reaction solution is described in Table 1.

TABLE 1

| (Final) concentration | | Ingredients |
|---|---|---|
| 2 | mg/mL | Trypsin |
| 1.2 | U/mL | Dispase |
| 1 | mg/mL | Type 1 collagenase |
| 20 | μg/mL | DNase 1 |

| | |
|---|---|
| — | HBSS (Hank's balanced salt solution) |

After adding 5% FBS (fetal bovine serum) to the culture, the mixture was centrifuged at 1,500 rpm for 5 minutes. After the centrifugation, the supernatant was removed and the remaining pellets were resuspended in 30 mL of DPBS and then centrifuged at 1,500 rpm for 5 minutes. After the centrifugation, the supernatant was removed and the remaining pellets were resuspended in 10 mL of DPBS to prepare a suspension. The suspension was passed through a 100-μm filter. The tonsil-derived mesenchymal stem cells remaining in the filter were washed with 20 mL of DPBS and then centrifuged at 1,500 rpm for 5 minutes. After the centrifugation, the supernatant was removed and incubation was performed in a constant-temperature water bath at 37° C. for 5 minutes after adding an ACK lysis buffer. After adding DPBS to the suspension, centrifugation was conducted at 1,500 rpm for 5 minutes. After the centrifugation, the supernatant was removed and the remaining pellets were resuspended in high-glucose DMEM (10% FBS, 20 μg/mL gentamicin) to prepare a cell suspension. Then, the number of cells in the prepared cell suspension was counted. The cell suspension was seeded in a T175 flask and incubated at 37° C. in a 5% CO₂ incubator.

### Example 1.2—Obtaining AD-MSC

Adipose-derived mesenchymal stem cells were purchased from Lonza (human adipose-derived stem cells, Cat #PT-5006, Lonza, Switzerland). The adipose-derived mesenchymal stem cells were cultured using a medium provided by Lonza (Bulletkit ADSD, Cat #PT-4505).

### Example 1.3 Measuring Population Doubling Times

As previously discussed the population doubling time refers to the time required for a population to double in size. After the MSC samples were obtained for culturing, the inventors used a flow cytometer to determine cell count data. The inventors then used the cell count data to calculate the population doubling time. Specifically, the inventors relied on the formula, Doubling Time (hours)=(t−t₀)×log(2)/log(Nₜ/N₀), where t is the time at the end of the measurement period, t₀ is the time at the beginning of the measurement period, Nₜ is the cell count at time t, and N₀ is the initial cell count at time to.

### Example 2. Formation of Neurospheres

Neurospheres were formed by culturing the mesenchymal stem cells of Example 1. Specifically, the mesenchymal stem cells were subcultured to 4-7 passages. After removing the culture medium, the mesenchymal stem cells were washed with DPBS. After treating the washed cells with TrypLE, the harvested cells were counted. After centrifuging the harvested cells and removing the supernatant, they were resuspended in a neurosphere formation medium. The composition of the neurosphere formation medium is described in Table 2.

TABLE 2

| (Final) concentration | | Ingredients |
|---|---|---|
| — | | DMEM/F12 with GlutaMAX |
| 20 | ng/ml | Basic fibroblast growth factor |
| 20 | ng/ml | Epidermal growth factor |
| 1x | | B27 supplement |
| 20 | μg/mL | Gentamicin |

The cells resuspended in the neurosphere (1×10⁶ cells) formation medium were seeded on an ultra-low attachment dish (60 mm). The seeded cells were cultured for 3 days under the condition of 37° C. and 5% CO₂. After the culturing for 3 days, the neurospheres formed on the dish were collected in a 15-mL tube. After centrifuging the collected cells and removing the supernatant, a neurosphere suspension was prepared by adding a fresh neurosphere formation medium. The neurosphere suspension was transferred to an ultra-low attachment dish and the neurospheres were cultured for 4 days under the condition of 37° C. and 5% CO₂.

### Example 3. Differentiation into Candidate Cells of Neuronal Regeneration-Promoting Cells (NRPCs) Using Neurospheres

The neurospheres formed in Example 2 were crushed finely using a 23-26G syringe needle. The crushed neurospheres were transferred to a 15-mL tube using a pipette and then centrifuged. After removing the supernatant, the crushed neurospheres were resuspended by adding a neuronal regeneration-promoting cell induction medium to the tube. Various neuronal regeneration-promoting cell induction media were prepared by combining three or more of 1) 5-20% FBS (fetal bovine serum), 2) 5-20 ng/mL bFGF (Peprotech, USA), 3) 100-400 μM butylated hydroxyanisole (Sigma, USA), 4) 5-40 μM forskolin (MedCheExpress, USA), 5) 0.1-10% N2 supplement (GIBCO, USA), 6) 1-100 ng/ml brain-derived neurotrophic factor (BDNF, Sigma-Aldrich, USA), 7) 1-100 ng/mL nerve growth factor (NGF, Santa Cruz, USA), 8) 0.01-1 ng/ml sonic hedgehog (SHH, R & D Systems, USA), 9) 1-10 ng/mL PDGF-AA (platelet-derived growth factor-AA, Peprotech, USA) and 10) 50-300 ng/mL heregulin-beta1, Peprotech, USA) in DMEM/F12 containing GlutaMAX.

The neurospheres resuspended in the various media were seeded onto a T175 flask coated with laminin (2 μg/mL). The seeded neurospheres were cultured for 8-10 days while exchanging the neuronal regeneration-promoting cell induction medium at 3-day intervals (FIG. 1).

### Example 4. Screening of Neuronal Regeneration-Promoting Cells Through Analysis of CD Marker Expression

The expression of a total of 242 CD markers was analyzed in the tonsil-derived mesenchymal stem cells (T-MSCs), where myelination was confirmed cytomorphologically among the neuronal regeneration-promoting cell candidates from Example 4, and neuronal regeneration-promoting cells differentiated therefrom.

For the analysis of CD markers, 3×10⁷ target cells were collected. The target cells were washed with DPBS and then centrifuged at 2000 rpm for 5 minutes. After removing the supernatant and washing once with DPBS, centrifugation was conducted and the remaining pellets were resuspended in 30 mL of a FACS buffer. 100 μL of the cell suspension (1×10⁵ cells) was seeded in each well of a round-bottomed 96-well plate. Then, 10 μL of primary antibodies of CD markers were added to each well of the 96-well plate. After reaction for 30 minutes on ice with the light blocked, each well was washed with 100 μL of a FACS buffer and then centrifugation was performed at 300 g for 5 minutes. After removing the supernatant and adding 200 μL of a FACS buffer to each well, centrifugation was performed at 300 g for 5 minutes. Secondary antibodies were prepared in a FACS buffer at a ratio of 1:200 (1.25 μg/mL). After the centrifugation was completed, the supernatant was removed and then 100 μL of the prepared secondary antibodies were added to each well. After reaction for 20-30 minutes on ice with the light blocked, each well was washed with 100 μL of a FACS buffer and then centrifugation was performed at 300 g for 5 minutes. After removing the supernatant, the target cells were washed by adding 200 μL of a FACS buffer to each well. The washing procedure was repeated twice. After the washing, the cells were resuspended by adding 200 μL of a FACS buffer to each well and the expression of CD markers in the target cells was investigated by flow cytometry or FACS (fluorescence-activated cell sorting).

The result of comparing the expression of CD markers in the induced neuronal regeneration-promoting cells using heat maps is shown in FIG. 3. As shown in FIG. 3, the neuronal regeneration-promoting cells (NRPCs) and the mesenchymal stem cells (MSCs) showed similar CD marker expression patterns but showed difference in the expression pattern of some markers. The CD marker expression pattern of the MSCs, and the respective NRPCs to which the MSCs differentiated into, were compared to select the CD markers for which the expression increased or decreased as differentiation markers for neuronal regeneration-promoting cells. The CD markers for which the expression in the NRPCs increased or decreased are shown in FIG. 3

As shown in FIG. 3, the CD markers for which the expression in the NRPCs increased (when compared to the respective MSCs from which the NRPCs were derived from, were CD106, CD112, CD121a, CD338, etc, and such expressions were most prominent for the tonsil derived NRPCs (T-NRPCs) differentiated from the tonsil-derived MSCs (T-MSCs). And, as shown in FIG. 3, the CD markers the expression of which has decreased included CD26 CD54, CD141, etc. Furthermore, as shown in FIG. 3, the expression of CD121a was particularly high in the T-NRPCs, as compared to other NRPCs. The effects of expression of CD121a was thus further investigated, as previously discussed, and was found to be particularly relevant for facilitating neurite outgrowth. From the above results, the CD markers the expression of which has increased or decreased commonly in the tonsil-derived neuronal regeneration-promoting cells (T-NRPCs) derived from T-MSCs were screened. The screened markers are as follows: The CD markers the expression of which has increased commonly: CD106, CD112 and CD121a. The CD markers the expression of which has decreased commonly: CD26 and CD141. The pattern of the CD markers the expression of which has increased or decreased commonly was observed identically also in the neuronal regeneration-promoting cells differentiated from the adipose-derived mesenchymal stem cells of Example 1-2. Thus, the CD markers—CD26 CD106, CD112, CD121a, and CD141—were found to be useful for identifying NRPCs differentiated from MSCs. While the markers CD121a, CD106, CD112, CD26 and CD141 expression of which has changed commonly can be used as differentiation markers of neuronal regeneration-promoting cells, the inventors discovered that high expression rates for CD121a is particularly useful for identifying effective NRPCs, as discussed herein.

### Example 4. Cytokine Array Assay of Neuronal Regeneration-Promoting Cells

As shown in FIG. 4A and FIG. 4B, the expression of 507 cytokines was analyzed in T-MSCs and AD-MSCs, and the respective NRPCs differentiated therefrom—T-NRPCs and AD-NRPCs. The target cells were cultured for analysis of the cytokines. The target cells were seeded in a flask and cultured for 3-4 days. When the target cells filled 80% or more of the area of the flask, the culture medium was removed and the target cells were washed twice with DPBS. After the washing, the culture medium was replaced with DMEM (Dulbecco's phosphate-buffered saline) not containing FBS (fetal bovine serum), cytokines, etc. in order to rule out the effect of cytokines. The culture of the target cells was collected after culturing for 30 hours.

The collected culture was centrifuged at 3,600 rpm for 30 minutes. The supernatant was transferred to a centrifugal tube equipped with a cellulose membrane and concentrated by centrifuging at 3,600 rpm for 20 minutes. After the centrifugation, the conditioned medium that passed through the separation membrane was discarded and the culture of the same amount was added. The centrifugation was continued until the volume of the concentrated culture was decreased to 1 mL or below, and the concentrated culture was quantified by Bradford assay. The concentrated culture was adjusted to a final concentration of 1 mg/mL by mixing with DMEM.

A membrane coated with antibodies capable of detecting 507 cytokines (cytokine array kit, RayBiotech, USA) was reacted for 30 minutes by treating with a blocking buffer. After removing the blocking buffer remaining on the membrane and replacing with the concentrated culture, the membrane was reacted overnight in a refrigerator. The membrane was washed 7 times with a washing buffer. After adding a HRP-conjugated streptavidin solution, the membrane was reacted at room temperature for 2 hours. After removing the HRP-conjugated streptavidin solution, the membrane was washed 7 times with a washing buffer. After the washing, the membrane was soaked with an ECL (enhanced chemiluminescence) reagent and the expression of cytokines was confirmed using an imaging device.

The result of comparing the expression of cytokines in the neuronal regeneration-promoting cells using the heat map shown in FIG. 4A and FIG. 4B. In FIG. 4A and FIG. 4B, the AD-MSCs, AD-NRPCs, T-MSCs, and T-NRPCs showed different expression patterns. As previously discussed, the cytokines HGF, uPA, and GRO-α, which are particularly relevant for nerve regeneration, were expressed higher in the NRPCs than the MSCs, and especially in the T-NRPCs.

### Example 5. Screening of Candidate Cells of Neuronal Regeneration-Promoting Cells Through Confirmation of Myelination of Peripheral Nerves

It was investigated whether the neuronal regeneration-promoting cell candidates prepared in Example 3 have the ability of myelinating peripheral nerves. Specifically, the differentiated neuronal regeneration-promoting cell candidates were co-cultured with dorsal root ganglia (DRG) and it was investigated whether myelination occurred.

Dorsal root ganglion (DRG) cells isolated from rats were purchased from Lonza (rat dorsal root ganglion cells, Cat #R-DRG-505, Lonza, Switzerland). The candidate cells were co-cultured with the purchased dorsal root ganglia. The DRG cells were cultured using a culture medium provided by Lonza (primary neuron growth medium bullet kit (PNGM), Cat #CC-4461).

The culture medium was exchanged every 3 days. As a result of co-culturing the candidate cells with the dorsal root ganglia, it was confirmed that myelination was achieved cytomorphologically in some of the cells (FIGS. 5-8B). Specifically, as previously discussed, the inventors determined that the number of axons and the length of the longest axon increased significantly in the assay groups using T-NRPCs in the culture medium compared to the assay groups using T-MSCs, AD-MSCs, or AD-NRPCs in the culture media.

### VARIOUS CHANGES AND MODIFICATIONS TO THE EXAMPLE EMBODIMENTS DESCRIBED HEREIN WILL BE APPARENT TO THOSE SKILLED IN THE ART

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. A plurality of cells induced from tonsil-derived mesenchymal stem cells and expressing CD26, CD106, CD112, CD121a, and CD141, wherein CD121a has an expression level of about 75% or higher.

2. The cells of claim 1, wherein the expression level of CD121a in the cells is measured during one or more passages after thawing from a frozen state.

3. The cells of claim 1, wherein the expression level of CD121a in the cells is about 90% or higher when measured during one or more passages after thawing from a frozen state.

4. The cells of claim 1,
wherein an expression level of CD26 in the cells is about 5% or lower,
wherein an expression level of CD106 in the cells is about 15% or higher,
wherein an expression level of CD112 in the cells is about 50% or higher, and
wherein an expression level of CD141 in the cells is about 30% or lower,
wherein the cells are in a thawed state after thawing from a frozen state without a subsequent passage, wherein the expression levels of CD26, CD106, CD112, and CD141 are measured in the thawed state.

5. The cells of claim 1,
wherein the expression level of CD26 in the cells is between about 10% and about 35%,
wherein the expression level of CD106 in the cells is between about 10% and about 35%,
wherein the expression level of CD112 in the cells is between about 25% and about 90%, and
wherein the expression level of CD141 in the cells is between about 10% and about 45%,
wherein the expression levels of CD26, CD106, CD112, and CD141 are measured in the thawed state.

6. A method of treating damaged nerve cells, the method comprising:
administration, into a subject's body having the damaged nerve cells, a composition comprising the cells of claim 1 in an effective amount for causing myelination of damaged nerve cells or remyelination of Schwann cells.

7. A method of treating muscle fibrosis, the method comprising:
administration, into a subject's body having the muscle fibrosis, a composition comprising the cells of claim 1 in an effective amount for treating the muscle fibrosis.

8. A method of treating muscle inflammation, the method comprising:
administration, into a subject's body having the muscle inflammation, a composition comprising the cells of claim 1 in an effective amount for treating the muscle inflammation.

9. A method of causing vascularization in an ischemic tissue, the method comprising:
administration, into a subject's body having an ischemic tissue, a composition comprising the cells of claim 1 in an effective amount for causing vascularization.

10. A method of treating critical limb ischemia (CLI), the method comprising:
injecting, into a subject's body having the CLI, a composition comprising the cells of claim 1 in an effective amount for treating the CLI.

11. A method of treating peripheral nerve damage, the method comprising:
administrating, into a subject's body having the peripheral nerve damage, a composition comprising the cells of claim 1 in an effective amount for treating the peripheral nerve damage.

12. A method of suppressing overexpression of peripheral myelin protein 22 (PMP22), the method comprising:
administering, into a localized area of a subject's body in which overexpression of PMP22 is confirmed or assessed, a composition comprising the cells of claim 1 in an effective amount for suppressing the overexpression of PMP22 at least in the localized area.

13. The method of claim 14, wherein the method treats Charcot-Marie-Tooth (CMT) in the subject.

14. The method of claim 14, wherein the composition is administered immediately after thawing the cells from a frozen state.

15. A method of increasing expression of miR-29a, the method comprising
administering, into a localized area of a subject's body in which a need for increasing expression of miR-29a is confirmed or assessed, a composition comprising the cells of claim 1 in an effective amount for increasing the expression of miR-29a at least in the localized area.

16. The method of claim 17, wherein increasing the expression of miR-29a causes suppressing overexpression of peripheral myelin protein 22 (PMP22) at least in the localized area.
